(19) United States
(12) Patent Application Publication
(10) Pub. No.: US 2024/0099887 A1
(43) Pub. Date: Mar. 28, 2024

(54) SYSTEM AND METHOD FOR LASER TREATMENT OF OCULAR TISSUE BASED ON PIGMENTATION

(71) Applicant: ViaLase, Inc., Aliso Viejo, CA (US)

(72) Inventors: Guy Holland, San Juan Capistrano, CA (US); Tibor Juhasz, San Clemente, CA (US); Reza Khazaeinezhad, Lake Forest, CA (US); Wesley W. Lummis, Rancho Santa Margarita, CA (US); Eric R. Mikula, Aliso Viejo, CA (US); Ferenc Raksi, Mission Viejo, CA (US); Manu Sharma, Ladera Ranch, CA (US); Hadi Srass, Yorba Linda, CA (US); Carlos G. Suarez, Tustin, CA (US)

(21) Appl. No.: 17/955,321

(22) Filed: Sep. 28, 2022

(51) Int. Cl. A61F 9/008

(52) U.S. Cl. CPC A61F 9/0084; A61F 2009/00844; A61F 2009/00891; A61F 2009/00897

(57) ABSTRACT

A system accesses a target volume of ocular tissue and treats the target volume with a laser based on the pigmentation of the tissue. A visual observation apparatus captures a color image of the target volume through an optics assembly. A control system processes the captured image and determines an energy parameter based on pigmentation of the target volume of ocular tissue. A laser source outputs a laser beam, and one or more components focus, scan, and direct the laser beam through the target volume by way of the optical assembly. The control system controls the one or more components based on a treatment pattern to place a focus of the laser beam at an initial location within the target volume of ocular tissue, and controls the laser source to apply photodisruptive energy at the initial location based on the energy parameter determined by the control system.

### TECHNICAL FIELD

The present disclosure relates generally to the field of medical devices and treatment of diseases in ophthalmology, and more particularly to systems and methods for laser treatment of tissue targets in the eye based on pigmentation of the tissue.

### BACKGROUND

Before describing the different types of glaucoma and current diagnosis and treatments options, a brief overview of the anatomy of the eye is provided.

Anatomy of the Eye

With reference to FIG. 1, the outer tissue layer of the eye 1 includes a sclera 2 that provides the structure of the eye's shape. In front of the sclera 2 is a cornea 3 that is comprised of transparent layers of tissue that allow light to enter the interior of the eye. Inside the eye 1 is a crystalline lens 4 that is connected to the eye by fiber zonules 5, which are connected to the ciliary body 6. Between the crystalline lens 4 and the cornea 3 is an anterior chamber 7 that contains a flowing clear liquid called aqueous humor 8. Encircling the perimeter of the crystalline lens 4 is an iris 9 which forms a pupil around the approximate center of the crystalline lens. The vitreous humor 10 is located between the crystalline lens 4 and the retina 11. Light entering the eye is optically focused through the cornea 3 and crystalline lens.

Referring to FIG. 2, as an optical system, the eye 1 is represented by an optical model described by idealized centered and rotationally symmetrical surfaces, entrance and exit pupils, and six cardinal points: object and image space focal points, first and second principal planes, and first and second nodal points. Angular directions relative to the human eye are often defined with respect to an optical axis 24, a visual axis 26, a pupillary axis 28 and a line of sight 29 of the eye. The optical axis 24 is the symmetry axis, the line connecting the vertices of the idealized surfaces of the eye. The visual axis 26 connects the foveal center 22 with the first and second nodal points to the object. The line of sight 29 connects the fovea through the exit and entrance pupils to the object. The pupillary axis 28 is normal to the anterior surface of the cornea 3 and is directed to the center of the entrance pupil. These axes of the eye differ from one another only by a few degrees and fall within a range of what is generally referred to as the direction of view.

Laser Surgery

Lasers, such as femtosecond lasers, are used and treatment of diseases in ophthalmology. For example, femtosecond lasers may be used in the treatment of glaucoma. Femtosecond laser pulses treat tissue by a process called photodisruption in which tissue at the focus of a beam is disrupted to elemental gas. The intent of treating the tissue in this manner is to create an aperture through which the intraocular pressure can be reduced.

The “cutting efficiency” is a function of laser fluence, which is the ratio of energy per pulse to the area over which the energy is delivered, spot size. Once the laser fluence exceeds a breakdown threshold value, the tissue within a volume specified by the laser focus spot size is disrupted. If the laser fluence is less than the breakdown threshold, the focused laser does not affect the tissue. It is generally accepted that the breakdown threshold for ocular tissue is approximately 0.8 to 1.2 J/cm². The breakdown threshold, however, is contingent upon patient specific anatomy, which influences the absorption of energy by the tissue. As a result, the “cutting efficiency” of a laser varies from patient to patient.

### SUMMARY

The present disclosure relates to a method of photodisrupting a target volume of ocular tissue of an eye with a laser. The method includes determining an energy parameter based on pigmentation of the target volume of ocular tissue; placing a focus of a laser at an initial location within the target volume of ocular tissue; and applying photodisruptive energy by the laser at the initial location based on the energy parameter. The pigmentation of the target volume of ocular tissue, from which an energy parameter is determined, may be based on numerical comparisons of colors present in pixels of a color image of the tissue and a reference color, with different energy parameters assigned to different numerical values. The pigmentation of the target volume of ocular tissue, from which an energy parameter is determined, may be based on algorithmic partitioning of pixels in a color image of the tissue into clusters of pixels, with different energy parameters assigned to different clusters.

The present disclosure also relates to an integrated surgical system for photodisrupting a target volume of ocular tissue of an eye with a laser. The system includes a laser source that is configured to output a laser beam; a visual observation apparatus that is configured to output a visual observation beam and to capture color images; a plurality of components that are optically coupled to receive the laser beam and the visual observation beam and that are configured to one or more of focus, scan, and direct the laser beam and the visual observation beam; and an optics assembly that is configured to couple to the eye and that is optically coupled to receive the laser beam and the visual observation beam and to direct the laser beam and the visual observation beam to the target volume of ocular tissue. The integrated surgical system further includes a control system that is coupled to the laser source, the visual observation apparatus, and one or more of the plurality of components. The control system is configured to: determine an energy parameter based on pigmentation of the target volume of ocular tissue, control one or more of the plurality of components to place a focus of a laser beam from the laser source at an initial location within the target volume of ocular tissue; and control the laser source to apply photodisruptive energy by the laser beam at the initial location based on the energy parameter.

The present disclosure also relates to a control system that controls the operation of a surgical system. The control system is coupled to a laser source configured to output a laser beam, a visual observation apparatus configured to output a visual observation beam and to capture color images, and one or more of a plurality of components coupled to receive the laser beam and the visual observation beam and configured to one or more of focus, scan, and direct the laser beam and the visual observation beam. The control system is configured to determine an energy parameter based on pigmentation of a target volume of ocular tissue, control one or more of the plurality of components to place a focus of a laser beam from the laser source at an initial location within the target volume of ocular tissue; and control the laser source to apply photodisruptive energy by the laser beam at the initial location based on the energy parameter.

It is understood that other aspects of apparatuses and methods will become apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of systems, apparatuses, and methods will now be presented in the detailed description by way of example, and not by way of limitation, with reference to the accompanying drawings, wherein:

FIG. 1 is a sectional schematic illustration of a human eye and its interior anatomical structures.

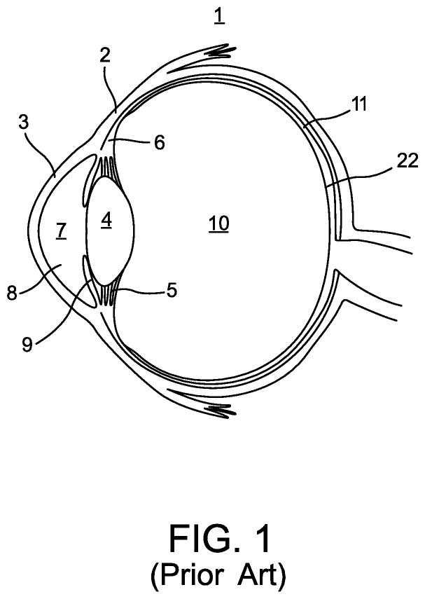

FIG. 2 is a sectional schematic illustration of a human eye showing various axes associated with the eye.

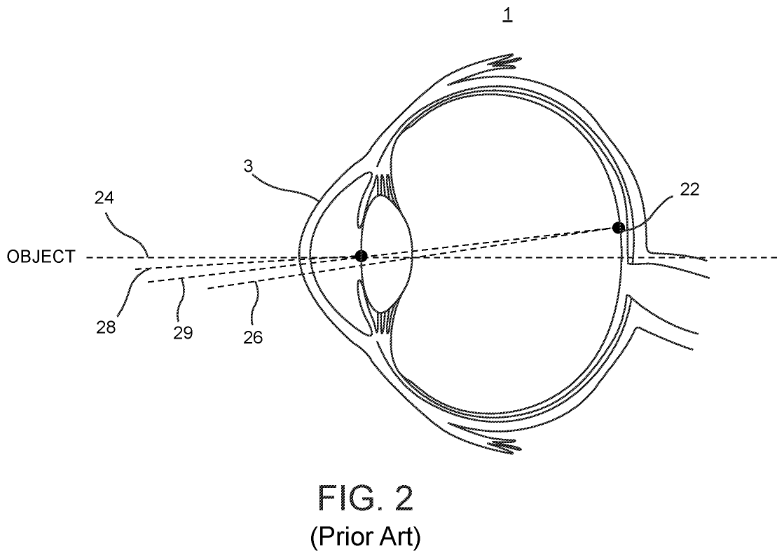

FIG. 3 is a sectional schematic illustration of light-beam paths to different tissue targets of the eye provided by an integrated surgical system for ophthalmic surgery disclosed herein.

FIG. 4 is a block diagram of an integrated surgical system for ophthalmic surgery.

FIG. 5A is a detailed block diagram of an integrated surgical system of FIG. 4 configured to deliver one or more light beams along a single beam path to a select tissue target of the eye.

FIG. 5B is a detailed block diagram of an integrated surgical system of FIG. 4 configured to deliver one or more light beams along a multiple beam paths to a select tissue target of the eye.

FIGS. 6A and 6B are schematic illustrations of an embodiment of an integrated surgical system having a focusing objective head with a symmetric optics assembly coupled to an eye through a patient interface.

FIGS. 7A and 7B are schematic illustrations of an embodiment of an integrated surgical system having a focusing objective head with an asymmetric optics assembly coupled to an eye through a patient interface.

FIGS. 8A, 8B, 8C, and 8D are schematic illustrations of other configurations of asymmetric optics assemblies that may be used in place of the optics assembly of FIG. 7A.

FIG. 8E includes isometric illustrations of an embodiment of an asymmetric optics assembly that may be used in place of the optics assembly of FIG. 7A.

FIG. 9 is a schematic illustration of an embodiment of an integrated surgical system having a focusing objective head rotatably coupled to an eye through an interface structure and a patient interface.

FIG. 10 is a schematic illustration of another embodiment of an integrated surgical system having a focusing objective head rotatably coupled to an eye through an interface structure and a patient interface.

FIG. 11 is a three-dimensional schematic illustration of the irido-corneal angle, including the trabecular meshwork, Schlemm's canal, a collector channel branching from the Schlemm's canal, and a target volume of ocular tissue to be treated by an integrated surgical system.

FIG. 12 is a two-dimensional schematic illustration of the irido-corneal angle with a target volume of ocular tissue, and a three-dimensional laser treatment pattern to be applied by an integrated surgical system to treat the target volume of ocular tissue.

FIG. 13 is a three-dimensional schematic illustration of FIG. 11 subsequent to treatment of the target volume of ocular tissue by a laser based on the laser treatment pattern of FIG. 12 to form an opening, aperture, or void through the trabecular meshwork.

FIG. 14A is a schematic illustration of a three-dimensional laser treatment pattern formed by a number of stacked two-dimensional treatment planes or layers.

FIG. 14B is a schematic illustration of a two-dimensional treatment layer defined by an array of spots.

FIG. 15 is a flowchart of a method of treating a target volume of ocular tissue in an eye based on tissue pigmentation.

FIG. 16A is a two-dimensional schematic illustration of an image of the irido-corneal angle of an eye that includes a target volume of ocular tissue for treatment in accordance with the method of FIG. 15.

FIG. 16B is a three-dimensional schematic illustration of the target volume of ocular tissue captured in the image of FIG. 16A.

FIG. 16C is a two-dimensional detailed schematic illustration of an array of pixels forming a color image of the target volume of ocular tissue of FIG. 16A.

### DETAILED DESCRIPTION

As previously noted, femtosecond laser pulses treat tissue by a process called photodisruption in which tissue at the focus of a beam is disrupted to elemental gas. The intent of treating the tissue in this manner is to create or cut an aperture through ocular tissue. For example, it may be desirable to create or cut an aperture through the irido-corneal angle, the cornea, the crystalline lens, the posterior capsule of the lens, the anterior capsule of the lens, the vitreous humor, and the retina.

The “cutting efficiency” of a laser treatment is a function of laser fluence, which is the ratio of energy per pulse to the area over which the energy is delivered. The area over which the energy is delivered is referred to as a laser focus spot size. Once the laser fluence exceeds a breakdown threshold value, the tissue within a volume specified by the laser focus spot size is disrupted. If the laser fluence is less than the breakdown threshold, the focused laser does not affect the tissue.

It is generally accepted that the breakdown threshold for ocular tissue is approximately 0.8 to 1.0 μJ/cm². The breakdown threshold, however, is also contingent upon the level of tissue pigmentation, which influences the absorption of energy by the tissue. For example, a trabecular meshwork that is more lightly pigmented requires more laser energy than more darkly pigmented trabecular meshwork. As a result, the “cutting efficiency” of the laser varies from patient to patient. Moreover, the cutting efficiency can vary within a single treatment since the pattern can be applied to tissues with varying pigmentation.

An integrated surgical system disclosed herein is configured to access a target volume of ocular tissue of an eye and photodisrupt the target volume of ocular tissue with a laser based on the pigmentation of the tissue. To this end, the integrated surgical system includes a visual observation apparatus that captures a color image of the target volume through an optics assembly that enables access to various tissue targets of the eye. The integrated surgical system includes a control system that processes the captured image and determines an energy parameter based on pigmentation of the target volume of ocular tissue. The integrated surgical system also includes a laser source that outputs a laser beam, and one or more components that focus, scan, and direct the laser beam through the target volume by way of the optical assembly. The control system of the integrated surgical system controls the one or more components based on a treatment pattern to place a focus of the laser beam at an initial location within the target volume of ocular tissue, and controls the laser source to apply photodisruptive energy at the initial location based on the energy parameter determined by the control system.

Accessing Targets in the Eye

In the following description, the term “beam” may—depending on the context—refer to one of a laser beam, an OCT beam, an illumination beam, a visual observation beam, dual aiming beams, or any other type of light beam. The term “colinear beams” refers to two or more different beams that are combined by optics of the integrated surgical system to share a same path to a same target location of the eye as they enter the eye. The term “non-colinear beams” refers to two or more different beams that have different paths into the eye. The term “co-targeted beams” refers to two or more different beams that have different paths into the eye but that target a same location of the eye. In colinear beams, the different beams may be combined to share a same path into the eye by dichroic or polarization beam splitters, and delivered along a same optical path through a multiplexed delivery of the different beams. In non-colinear beams, the different beams are delivered into the eye along different optical paths that are separated spatially or by an angle between them. In the description to follow, any of the foregoing beams or combined beams may be generically referred to as a light beam. The terms distal and proximal may be used to designate the direction of travel of a beam, or the physical location of components relative to each other within the integrated surgical system. The distal direction refers to a direction toward the eye. The proximal direction refers to a direction away from the eye.

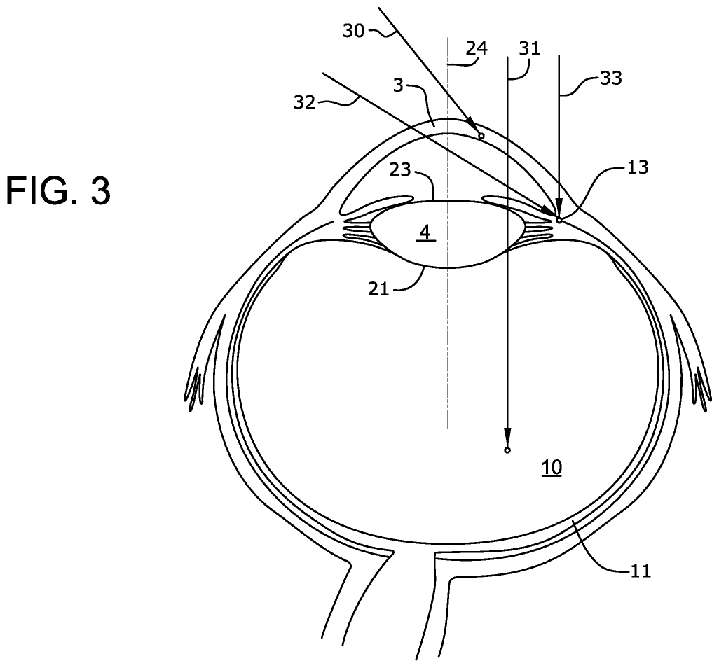

With reference to FIG. 3, a feature provided by the integrated surgical system disclosed herein is access to and visualization and image processing of different tissue targets in the eye. Different tissue targets include, for example, the irido-corneal angle 13, the cornea 3, the crystalline lens 4, the posterior capsule 21 of the lens, the anterior capsule 23 of the lens, the vitreous humor 10, and the retina 11.

A tissue target may be accessed by one or more lights beams along a single beam path. For example, one or more of a laser beam, an imaging beam, and a visual observation beam may access the cornea 3 along a first beam path 30. Given the angular arrangement of the first beam path 30 relative to the optical axis 24 of the eye, the first beam path may be referred to as an angled beam path. In another example, one or more of a laser beam, an imaging beam, and a visual observation beam may access the vitreous humor 10 along a second beam path 31 that is substantially parallel relative to the optical axis 24 of the eye. Given this alignment of the second beam path 31, it may be referred to as a parallel beam path. Substantially parallel means within 20 degrees of parallel. In some embodiments, a target may be accessed by different light beams along different, non-colinear beam paths 32, 33. For example, the irido-corneal angle 13 of the eye may be accessed by one or more beams along an angled beam path 32 passing through the cornea 3 and through the aqueous humor 8 in the anterior chamber 7, while one or more other beams may access the irido-corneal angle 13 along a parallel beam path 33 passing through the cornea 3 and into the irido-corneal angle 13 of the eye without passing through the aqueous humor 8 in the anterior chamber 7.

Integrated Surgical System

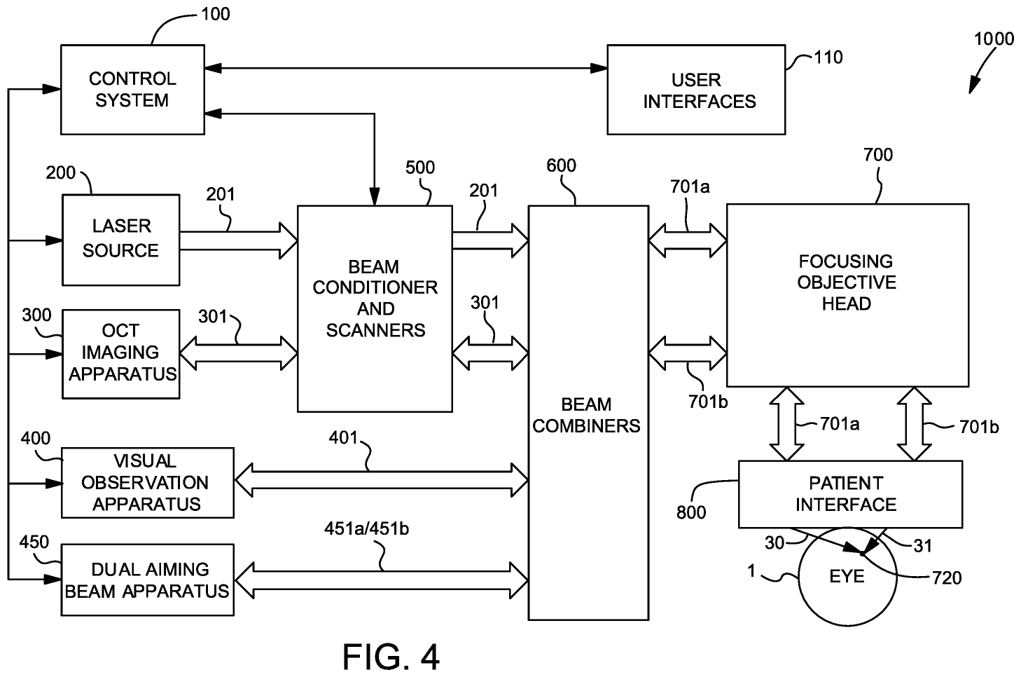

With reference to FIG. 4, in some embodiments the integrated surgical system 1000 for ophthalmic laser surgery disclosed herein includes a control system 100, one or more user interfaces 110, a surgical component 200, one or more imaging/visual components 300, 400, and a target locating apparatus 450. Other components of the integrated surgical system 1000 include beam conditioners and scanners 500, beam combiners 600, and a focusing objective head 700 that couples with a patient interface 800.

The surgical component 200 may be a femtosecond laser source that outputs a laser beam 201. A femtosecond laser provides highly localized, non-thermal photodisruptive laser-tissue interaction with minimal collateral damage to surrounding ocular tissue. Photodisruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam. The process can also be used in weakly absorbing or weakly scattering tissue. While femtosecond lasers with photodisruptive interactions have been successfully used in ophthalmic surgical systems and commercialized in other ophthalmic laser procedures, none have been used in an integrated surgical system that accesses the irido-corneal angle.

A first imaging/visual component 300 may be an OCT imaging apparatus that outputs an OCT beam 301. OCT technology provides imagery that assist in diagnosing, locating, and guiding laser surgery directed to different tissue targets in the eye. For example, with reference to FIG. 3, OCT imaging may be used to determine the structural and geometrical conditions of the irido-corneal angle 13 and to determine the accessibility of the ocular tissue for treatment. OCT imaging can provide the necessary spatial resolution, tissue penetration and contrast to resolve microscopic details of ocular tissue. When scanned, OCT imaging can provide two-dimensional (2D) cross-sectional images of the ocular tissue. As another aspect of the integrated surgical system, 2D cross-sectional images may be processed and analyzed to determine the size, shape, and location of structures in the eye for surgical targeting. It is also possible to reconstruct three-dimensional (3D) images from a multitude of 2D cross-sectional images but often it is not necessary. Acquiring, analyzing, and displaying 2D images is faster and can still provide all information necessary for precise surgical targeting.

A second imaging/visual component 400 may a visual observation apparatus that outputs a visual observation beam 401 and an illumination source. The visual observation apparatus 400 provides imagery that assist in identifying surgical locations. The visual observation apparatus may include, for example, a video camera and a telescope. The camera may be a digital camera fitted with a goniolens to provide gonioscopic images of the eye. The digital camera includes an image sensor or detector, typically a charge-coupled device (CCD) or a complementary metal-oxide-semiconductor (CMOS), comprising a two-dimensional array of pixels. The illumination source is positioned for optimal irradiance of the object of interested, e.g., tissue targets in the eye. Illumination sources may be LEDs or light delivered via fiber optic cables. Illumination schemes are numerous: refractive ballistic schemes where the sources are placed in air and light refracts through the optics to reach the trabecular meshwork; transmissive ballistic schemes where illumination sources are inserted into pre-drilled holes or features inside lenses and adhered using index-matched epoxy; or reflective schemes where light from illumination sources strikes the trabecular meshwork after reflecting off designed reflective surfaces on lenses close to the eye.

The target locating apparatus 450 may be a dual aiming beam apparatus such as disclosed in U.S. Patent Application Publication No. 2021/0235986, title “System and Method for Locating a surface of Ocular Tissue for Glaucoma Surgery Based on Dual Aiming Beams,” the contents of which are incorporated herein by reference. The dual aiming beam apparatus 450 outputs a pair of beams of light, referred to herein as dual aiming beams 451a/451b, for use in detecting a surface of ocular tissue in a surgical field.

The beam conditioner and scanners 500 are configured to set beam parameters of light beams including beam size and divergence. Beam conditioning may also include additional functions, such as setting the beam power or pulse energy and shutter the beam to turn it on or off. As shown in FIG. 4, a laser beam 201 from the femtosecond laser source 200 and an OCT beam 301 from the OCT imaging apparatus 300 are directed towards the beam conditioners and scanners 500. The beam conditioners and scanners 500 include components, e.g., scanning mirrors, for scanning the laser beams 201 and OCT beams 301 independent of each other. Different kind of scanners can be used for the purpose of scanning the laser beam 201 and the OCT beam 301. For scanning transversal to a light beam 201, 301, angular scanning galvanometer scanners are available for example from Cambridge Technology, Bedford, MA, Scanlab, Munich, Germany. To optimize scanning speed, the scanner mirrors are typically sized to the smallest size, which still support the required scanning angles and numerical apertures of the beams at the target locations. The ideal beam size at the scanners is typically different from the beam size of the laser beam 201 or the OCT beam 301, and different from what is needed at the entrance of a focusing objective head 700. Therefore, beam conditioners are applied before, after, or in between individual scanners.

The beam combiners 600 are configured to split and combine light beams. The beam combiners 500 may include dichroic or polarization beam splitters that split and recombine light beams with different wavelength and/or polarization. The beam combiner 600 may also include optics to change certain parameters of the individual light beams such as beam size, beam angle and divergence. As shown in FIG. 4, two or more of the laser beam 201, the OCT beam 301, the visual observation beam 401, and the dual aiming beams 451a/451b may be combined with dichroic, polarization or other kind of beam combiners 600 and provided to the focusing objective head 700 as a combined beam 701 to reach a common target volume of ocular tissue of the eye 1. For example, with reference to FIG. 3, in some embodiments all of these light beams 201, 301, 401, 451a/451b may be combined to reach a common target along a common beam path 30, 31. In some embodiments different sets of light beams may be combined to reach a common target along different beam paths 30, 31. For example, a first set of beams comprising the laser beam 201, the visual observation beam 401, and the dual aiming beams 451a/451b may be combined to reach the irido-corneal angle 13 along a first beam path 30, while a second set of beams comprising the OCT beam 301 may reach the irido-corneal angle along a second beam path 31.

Implementation of the functions of the beam conditioners and scanners 500 and beam combiners 600 may happen in a different order than what is indicated in FIG. 4. Specific optical hardware that manipulates the beams to implement those functions can have multiple arrangements with regards to how the optical hardware is arranged. They can be arranged in a way that they manipulate individual light beams separately, in another embodiment one component may combine functions and manipulate different beams. In the embodiment disclosed herein, the beam conditioners and scanners 500 include two sets of scanners, one for scanning the laser beam 201 and the other for scanning the OCT beam 301. Separate beam conditioners within the beam conditioners and scanners 500 set respective beam parameters for the laser beam 201 and the OCT beam 301.

The focusing objective head 700 includes one or more optical transmission subsystems optically coupled to receive one or more light beams 701a, 701b from the beam combiner 600. Going forward, a light beam identified by reference number 701a or 701b, may be an individual beam such as a laser beam 201, an OCT beam 301, a visual observation beam 401, dual aiming beams 451a/451b or any other type of light beam, or a combination of two or more of these individual beams. The focusing objective head 700 also includes an optics assembly. The optical transmission subsystems are configured to direct received light beams 701a, 701b into alignment with one of a plurality of input axes of the optics assembly. The optical transmission subsystems may be further configured for axial scanning of one or more light beams. The optics assembly is configured to direct a light beam it receives along an input axes to a corresponding output axis that is aligned with one or more tissue targets of the eye. Further details of the optical transmission subsystems and optics assembly are provided below.

The control system 100 is connected to the other components 200, 300, 400, 450, 500, 700 of the integrated surgical system 1000. Control signals from the control system 100 to the femtosecond laser source 200 function to control internal and external operation parameters of the laser source, including for example, power, repetition rate and beam shutter. Control signals from the control system 100 to the OCT imaging apparatus 300 function to control OCT beam parameters, and the acquiring, analyzing, and displaying of OCT images of tissue in the surgical field.

Control signals from the control system 100 to the dual aiming beam apparatus 450 function to control the output of beams of light by the one or more aiming beam sources of the dual aiming beam apparatus. Control signals from the control system 100 to the visual observation apparatus 400 function to control the capturing, image processing and displaying of video images of tissue in the surgical field and spots of light on tissue surfaces in the surgical field that result from the one or more beams of light output by the dual aiming beam apparatus 450. To this end, the line of sight of the visual observation apparatus 400 may be aligned with the femtosecond laser and directed into the target area of the eye.

Control signals from the control system 100 to the beam conditioner and scanners 500 function to control the scanning of a laser beam output by the femtosecond laser source 200 and the scanning of an OCT beam output by the OCT imaging apparatus 300. Control signals to the beam conditioner and scanners 500 may include location, size and shape of surgical patterns expressed in position coordinates of the intended location of focus of the laser and the scanning path of the laser across the surgical volume. These types of control signals can be pre-programmed, with one or more control parameters selectable by the operator. The control parameters of the surgical pattern may include the location of the pattern, the shape, length, width and depth of the pattern, laser spot, line and layer separation and energy of the laser pulses. Control signals to and from various subsystems and components are calibrated prior to operating the surgical system. The calibration includes calibrating the pixel coordinates acquired and displayed by the visual observation apparatus 400 and the OCT imaging apparatus 300 to actual physical coordinates in the eye and includes calibrating commanded motions of the OCT and laser scanner systems to actual OCT and laser beam displacements in the eye.

Control signals from the control system 100 to the focusing objective head 700 may function to control axial scanning of either or both of a laser beam 201 and an OCT beam 301 through a motorized focusing objective. Control signals from the control system 100 to the focusing objective head 700 may also function to control mechanical elements of one or more optical transmission subsystems to thereby direct one or more light beams 701a, 701b into alignment with an input axis of the optics assembly. Further details of the optical transmission subsystems and optics assembly are provided below.

Commanding the integrated surgical system 1000 to make a surgical incision includes docking the system on the eye, acquiring, and displaying visual observation images including spots from the dual aiming beams, and OCT images on a computer screen, determining the coordinate location and other parameters of the intended surgical incision based on the displayed images and instructing the control system 100 to execute the surgical pattern based on information collected from those images. The parameters based on the images may be determined by the operator of the integrated surgical system 1000 or may be determined by an image processing and analyzing computer algorithm. Instructions using these parameters can be given by the operator as entering input data in the form of text, mouse clicks and drag and drop commands on the computer screen. Alternatively, a system processor that may be included in the control system 100 generates instructions for execution by the control system based on the previously determined parameters.

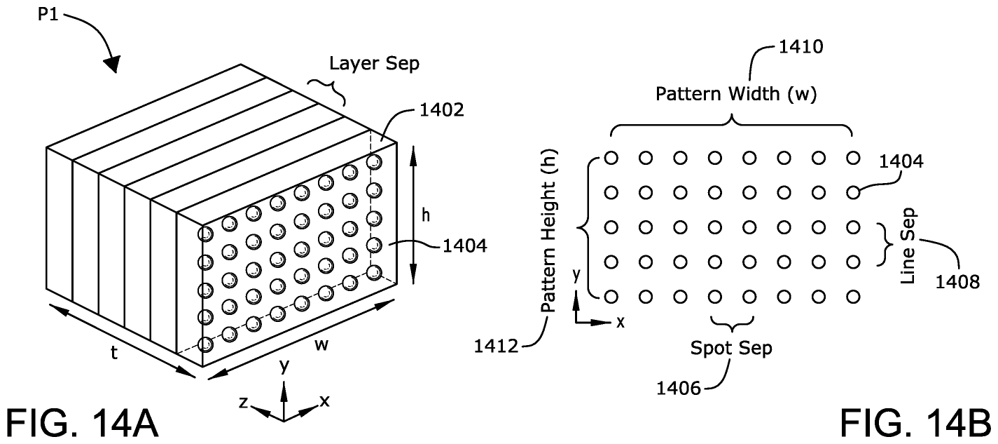

In accordance with embodiments disclosed herein, the control system 100 is configured to determine an energy parameter based on pigmentation of a target volume of ocular tissue and to control laser treatment of the volume based on the energy parameter. To this end, the control system 100 includes an image capture module that obtains a color image of the target volume. The color image may be obtained by the image capture module, for example, from the visual observation apparatus 400. The control system 100 also includes a pigmentation processing module that processes the captured image. Details of the image processing performed by the pigmentation processing module are described below with reference to FIG. 15. For now, in general terms, the pigmentation processing module is configured to execute an algorithm that determines a composite pigmentation metric for the target volume of ocular tissue based on comparisons of colors present in the color image and a reference color, and determines the energy parameter based on the composite pigmentation metric.

Single Optical Transmission Subsystem

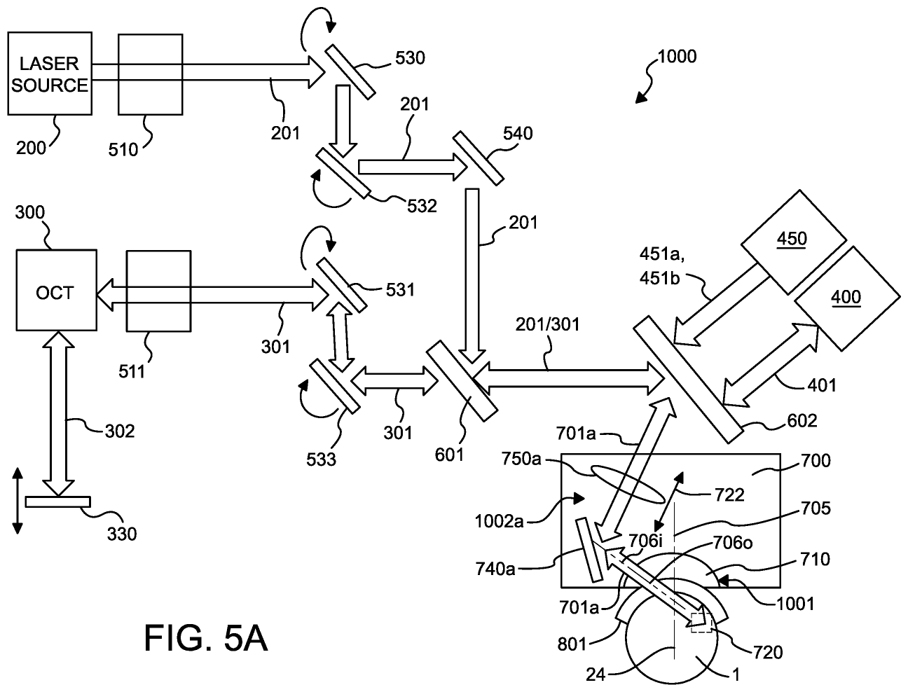

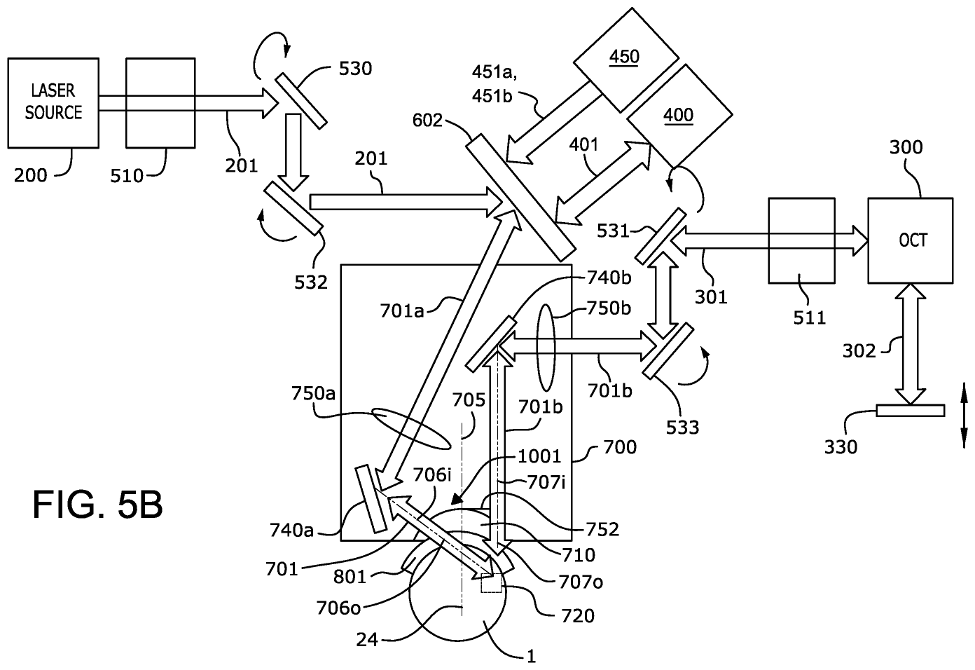

Referring to FIG. 5A, in accordance with embodiments disclosed herein an integrated surgical system may include a single optical transmission subsystem and an optics assembly configured to deliver one or more of a laser beam 201, an OCT beam 301, a visual observation beam 401, and a pair of aiming beams of light 451a, 451b in the distal direction toward an eye 1 along a single beam path, and to receive one or more of an OCT return beam 301 and a visual observation reflection beam 401 back from the eye 1 along the single beam path. In the example embodiment of FIG. 5A, the single beam path is into a target volume 720 of ocular tissue of the eye in the irido-corneal angle. In accordance with embodiments disclosed above with reference to FIG. 3, the single beam path may be into other target volumes 720 of ocular tissue of the eye including the cornea, the crystalline lens, the posterior capsule of the lens, the anterior capsule of the lens, the vitreous humor, and the retina.

Regarding the delivery of a laser beam, a laser beam 201 output by the femtosecond laser source 200 passes through a beam conditioner 510 where the basic beam parameters, beam size, divergence are set. The beam conditioner 510 may also include additional functions, setting the beam power or pulse energy and shutter the beam to turn it on or off. After existing the beam conditioner 510, a pair of transverse scanning mirrors 530, 532 rotated by a galvanometer scanner scan the laser beam 201 in two essentially orthogonal transversal directions, e.g., in the x and y directions. Then the laser beam 201 is directed towards a dichroic or polarization beam splitter 540 where it is reflected toward a beam combining mirror 601 configured to combine the laser beam 201 with an OCT beam 301.

Regarding delivery of an OCT beam, an OCT beam 301 output by the OCT imaging apparatus 300 passes through a beam conditioner 511 and a transversal scanner with scanning mirrors 531 and 533. Proceeding in the distal direction toward the eye 1, after the scanning mirrors 531 and 533, the OCT beam 301 is combined with the laser beam 201 by the beam combiner mirror 601. The OCT beam 301 and laser beam 201 components of the combined laser/OCT beam 210/301 are multiplexed and travel in the same direction. The combined laser/OCT beam 210/301 propagates to a second beam combining mirror 602 where it is combined with one or more aiming beams of light 451a/451b and a visual observation beam 401 to form a combined laser/OCT/visual/aiming beam 701a.

The combined light beam 701a (hereinafter referred to as a first light beam) traveling in the distal direction passes through a focusing objective 750a and is reflected by an alignment mechanism 740a, e.g., a beam-folding mirror, into alignment with an input axis 706ᵢ of an exit lens 710 of an optics assembly 1001. The alignment mechanism 740a and the focusing objective 750a are components of an optical transmission subsystem 1001a that may move the first light beam 701a into alignment with any one of a number of input axes 706ᵢ of the optics assembly 1001. The first light beam 701a passes through the exit lens 710 and exits the exit lens along an output axis 706o and into and through a window 801 of a patient interface into a focal point in the target volume 720. The focusing objective 750a, which may include a single lens or a group of lenses, is movable in the axial direction 722 by a servo motor, stepper motor or other control mechanism. Movement of the focusing objective 750a in the axial direction 722 changes the axial distance of the focus of the laser beam 201 and the OCT beam 301 at a focal point.

A scattered OCT return beam 301 from the target volume 720 of ocular tissue travels in the proximal direction to return to the OCT imaging apparatus 300 along the same paths just described, in reverse order. The reference beam 302 of the OCT imaging apparatus 300, passes through a reference delay optical path and return to the OCT imaging apparatus from a moveable mirror 330. The reference beam 302 is combined interferometrically with the OCT return beam 301 on its return within the OCT imaging apparatus 300.

Multiple Optical Transmission Subsystems

Referring to FIG. 5B, in accordance with embodiments disclosed herein an integrated surgical system may include multiple optical transmission subsystems and an optics assembly configured to deliver one or more of a laser beam 201, an OCT beam 301, a visual observation beam 401, and a pair of aiming beams of light 451a, 451b in the distal direction toward an eye 1, along either of multiple beam paths and to receive one or more of an OCT return beam 301 and a visual observation reflection beam 401 back from the eye 1 along either of multiple beam paths. In the example embodiment of FIG. 5B, the laser beam 201 and the visual observation beam 401 are delivered along a first optical path or first beam path into a region of the eye 1 that includes a target volume 720, while the OCT beam 301 is delivered along a second optical path or second beam path into the same region of the eye. The laser beam 201 and the OCT beam 301 are thus co-targeted, non-colinear beams. As shown in FIG. 5B, the first beam path and the second beam path are into a target volume 720 of ocular tissue of the eye in the irido-corneal angle. In accordance with embodiments disclosed above with reference to FIG. 3, the laser beam 201 and the OCT beam 301 may be co-targeted along different beam paths into other target volumes 720 of ocular tissue of the eye including the cornea, the crystalline lens, the posterior capsule of the lens, the anterior capsule of the lens, the vitreous humor, and the retina. Alternatively, the laser beam 201 and the OCT beam 301 may be targeted along different beam paths into different target volumes 720.

Regarding the delivery of a laser beam, a laser beam 201 output by the femtosecond laser source 200 passes through a beam conditioner 510 where the basic beam parameters, beam size, divergence are set. The beam conditioner 510 may also perform additional functions, such as setting the beam power or pulse energy and shuttering the beam to turn it on or off. After existing the beam conditioner 510, the laser beam 210 enters a pair of transverse scanning mirrors 530, 532 rotated by a galvanometer scanner scan the laser beam 201 in two essentially orthogonal transversal directions, e.g., in the x and y directions. Then the laser beam 201 is directed towards a beam combining mirror 602 configured to combine the laser beam 201 with a visual observation beam 401 and dual aiming beams 451a/451b to form a combined laser/OCT/visual/aiming beam 701a.

The combined light beam 701a (hereinafter referred to as a first light beam) traveling in the distal direction passes through a first focusing objective 750a and is directed by a first alignment mechanism 740a, e.g., a beam-folding mirror, into alignment with a first input axis 706ᵢ of an exit lens 710 of an optics assembly 1001. The first alignment mechanism 740a and the first focusing objective 750a are components of a first optical transmission subsystem 1002a that may move the first light beam 701a into alignment with any one of a number of first input axes 706ᵢ of the optics assembly 1001. The first light beam 701a passes through the exit lens 710 and exits the exit lens along a first output axis 706o and into and through a window 801 of a patient interface into a focal point in the target volume 720. The first focusing objective 750a, which may include a single lens or a group of lenses, is movable in the axial direction 722 by a servo motor, stepper motor or other control mechanism. Movement of the first focusing objective 750a in the axial direction 722 changes the axial distance of the focus of the laser beam 201 component of the first light beam 701a at a focal point.

Regarding delivery of an OCT beam, an OCT beam 301 output by the OCT imaging apparatus 300 passes through a beam conditioner 511 and a transversal scanner with scanning mirrors 531, 533. Proceeding in the distal direction toward the eye 1, after the scanning mirrors 531 and 533, the OCT beam 301 (hereinafter referred to as a second light beam) passes through a second focusing objective 750b and is directed by a second alignment mechanism 740b, e.g., a beam-folding mirror, into alignment with a second input axis 707ᵢ of a prism 752 of the optics assembly 1001. The second alignment mechanism 740b and the second focusing objective 750b are components of a second optical transmission subsystem 1002b that may move the second light beam 701b into alignment with any one of a number of second input axes 707ᵢ of the optics assembly 1001. The second light beam 701b then passes through the prism 752 and the exit lens 710 of the optics assembly 1001 and exits the exit lens along a second output axis 707o and into and through the window 801 of the patient interface into a focal point in the target volume 720. The second focusing objective 750b, which may include a single lens or a group of lenses, is movable in the axial direction 723 by a servo motor, stepper motor or other control mechanism. Movement of the second focusing objective 750b in the axial direction 723 changes the axial distance of the focus of the OCT beam 301 at a focal point.

A scattered OCT return beam 301 from the ocular tissue travels in the proximal direction to return to the OCT imaging apparatus 300 along the same paths just described, in reverse order. The reference beam 302 of the OCT imaging apparatus 300, passes through a reference delay optical path and return to the OCT imaging apparatus from a moveable mirror 330. The reference beam 302 is combined interferometrically with the OCT return beam 301 on its return within the OCT imaging apparatus 300.

Symmetric Optics Assemblies

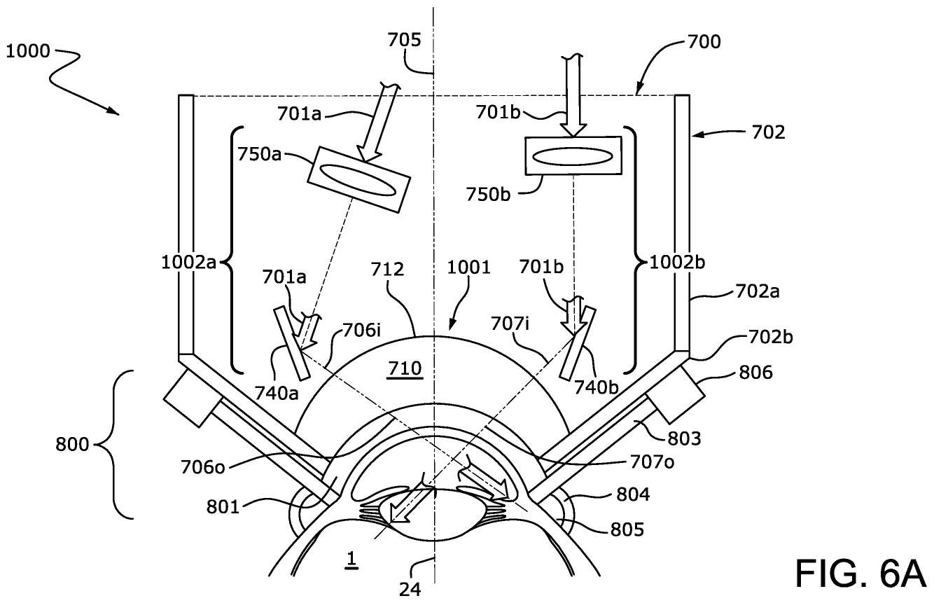

FIGS. 6A and 6B are schematic illustrations of an embodiment of a focusing objective head 700 of an integrated surgical system 1000 having a symmetric optics assembly 1001 optically coupled with a first optical transmission subsystem 1002a and an optional second optical transmission subsystem 1002b, each of which are optically coupled with respective components (not shown) to receive a first light beam 701a and a second light beam 701b. The optics assembly 1001 is mechanically coupled to a non-rotatable distal portion 702b of a housing 702 of the focusing objective head 700 while one or more components of the first optical transmission subsystem 1002a and the optional second optical transmission subsystem 1002b are mechanically coupled to a rotatable proximal portion 702a of the housing. Thus, in this embodiment the first optical transmission subsystem 1002a and the second optical transmission subsystem 1002b (if present) may be rotated relative to the optics assembly 1001, which remains fixed in place relative to the eye. While details of the mechanical coupling of the optics assembly 1001, the first optical transmission subsystem 1002a and the second optical transmission subsystem 1002b to the housing 702 are not illustrated, various means or mechanisms may be used to secure these components to the interior of the housing at appropriate locations to maintain their respective optical couplings.

Continuing with the embodiment of FIGS. 6A and 6B, the optics assembly 1001 includes an exit lens 710. The first optical transmission subsystem 1002a includes a first focusing objective 750a and a first alignment mechanism 740a and is optically coupled to receive the first light beam 701a incident a surface 712 of the exit lens 710 along a first input axis 706ᵢ. The surface 712 of the exit lens 710 to which the first light beam 701a is incident may be referred to herein an incident surface of the optics assembly 1001 or an entry surface of the optics assembly. The exit lens 710 directs the first light beam 701a into alignment with a first output axis 706ₒ of the optics assembly 1001. The first light beam 701a may be a laser beam, an OCT beam, a visual observation beam, dual aiming beams, or any other type of light beam or a combination thereof. The optional second optical transmission subsystem 1002b includes a second focusing objective 750b and a second alignment mechanism 740b and is optically coupled to receive a second light beam 701b incident a surface of the exit lens 710 along a second input axis 707ᵢ. The exit lens 710 directs the second light beam 701b into alignment with a second output axis 707ₒ of the optics assembly 1001. The second light beam 701b may be a laser beam, an OCT beam, a visual observation beam, dual aiming beams, or any other type of light beam or a combination thereof.

The optics assembly 1001 has an optical axis 705. The optics assembly 1001 is configured to couple to the eye through a patient interface 800 to align its optical axis 705 with the optical axis 24 of the eye. The optics assembly 1001 is optically coupled with the first optical transmission subsystem 1002a to receive the first light beam 701a along one of a plurality of first input axes 706ᵢ incident an incident surface 712 of the optics assembly and to direct the light beam through the optics assembly to a corresponding one of a plurality of first output axes 706ₒ aligned with a corresponding one of the plurality of target volumes of ocular tissue in the eye.

With reference to FIG. 6B (wherein for clarity of illustration, the optional second optical transmission subsystem 1002b is not shown), in some configurations the first alignment mechanism 740a of the first optical transmission subsystem 1002a comprises a means to direct the first light beam 701a into alignment with a select one of first input axes 7061 of the optics assembly 1001. To this end, the first alignment mechanism 740a is positioned and oriented relative to the incident surface 712 of the optics assembly 1001 and is configured to be repositioned and/or reoriented relative to the incident surface to direct the first light beam 701a into alignment with the selected first input axis 706ᵢ of the optics assembly.

In some embodiments the first alignment mechanism 740a may comprise an adjustable reflecting surface optically aligned to receive the first light beam 701a along an angle of incidence and to reflect the light beam at an angle of reflection into alignment with the corresponding one of the plurality of first input axes 706ᵢ of the optics assembly 1001. The reflecting surface may be for example, an actuated flip mirror or an actuated angular deflector that is repositioned and/or reoriented through a control signal from a control system 100.

In some embodiments the first alignment mechanism 740a may comprise a fiber-optic cable and a positioning mechanism. The fiber-optic cable is configured to transmit light and has a light-input end coupled to receive the first light beam 701a and a light-output end configured to output the first light beam. The positioning mechanism is mechanically coupled to the fiber-optic cable and is configured to reposition and/or reorient the light-output end into alignment with the selected first input axis 706ᵢ of the optics assembly in response to a control signal from a control system 100.

With continued reference to FIG. 6B, in other configurations the first focusing objective 750a of the first optical transmission subsystem 1002a may be configured to move relative to the first alignment mechanism 740a to direct the first light beam 701a into alignment with a select one of first input axes 706ᵢ of the optics assembly 1001. To this end, the first focusing objective 750a is positioned and oriented relative to the first alignment mechanism 740a and is configured to be mechanically repositioned and/or reoriented relative to the first alignment mechanism to direct the first light beam 701a into the first alignment mechanism at an angle of incidence that aligns the first light beam with the selected first input axis 706ᵢ of the optics assembly. For example, the first focusing objective 750a may be mounted for rotational movement relative to a reflecting surface of the first alignment mechanism 740a. A control signal from the control system 100 controls the position and orientation of the first focusing objective 750a.

In other configurations, both of the first alignment mechanism 740a and the first focusing objective 750a may be configured to move to direct the first light beam 701a into alignment with a select one of first input axes 706ᵢ of the optics assembly 1001. In the foregoing example embodiments of FIG. 6B, three different first input axes 706ᵢ of the optics assembly 1001 are illustrated. It is understood, however, that the number of first input axes 706ᵢ is not limited to three and may essentially comprise an almost infinite number of axes.

With reference to FIG. 6A, the optics assembly 1001 is optically coupled with the second optical transmission subsystem 1002b (if present) to receive the second light beam 701b along one of a plurality of second input axes 707ᵢ incident the incident surface 712 of the optics assembly and to direct the second light beam to a corresponding one of a plurality of second output axes 707ₒ aligned with a corresponding one of the plurality of target volumes of ocular tissue in the eye. The second optical transmission subsystem 1002b may be configured the same as the first optical transmission subsystem 1002a as described above. In other words, the second alignment mechanism 740b may be positioned and oriented relative to the incident surface 712 of the optics assembly 1001 and configured to be repositioned and/or reoriented relative to the incident surface to direct the second light beam 701b into alignment with the selected second input axis 707ᵢ of the optics assembly. Likewise, the second focusing objective 750b may be positioned and oriented relative to the second alignment mechanism 740b and configured to be mechanically repositioned and/or reoriented relative to the second alignment mechanism to direct the second light beam 701b into the second alignment mechanism at an angle of incidence that aligns the second light beam with the selected second input axis 707ᵢ of the optics assembly 1001.

In the embodiment of FIGS. 6A and 6B, components of the first optical transmission subsystem 1002a and the second optical transmission subsystem 1002b (if present) are configured to rotate about the optical axis 705 of the optics assembly 1001 to align with tissue targets of the eye at different locations around the circumferential angle of the eye, while the optics assembly itself does not rotate and thus remains fixed in place relative to the eye. To this end, as previously disclosed, the optics assembly 1001 is coupled to a non-rotatable distal portion 702b of the housing 702 of the focusing objective head 700, while one or more components of the first optical transmission subsystem 1002a, e.g., the first alignment mechanism 740a and/or the first focusing objective 750a, are mechanically coupled to a rotatable proximal portion 702a of the housing and arranged relative to other components (e.g., beam conditioners and scanners 500 and beam combiners 600) to maintain optical coupling with these components to receive the first light beam 701a regardless of the rotational position of the proximal portion of the housing. Similarly, if a second optical transmission subsystem 1002b is present, one or more components of the second optical transmission subsystem 1002b, e.g., the second alignment mechanism 740b and the second focusing objective 750b, may be mechanically coupled to the rotatable proximal portion 702a of the housing 702 and arranged relative to other components (e.g., beam conditioners and scanners 500 and beam combiners 600) to maintain optical coupling with these components to receive the second light beam 701b regardless of the rotational position of the proximal portion of the housing.

The optics assembly 1001 provides an interface to the eye through a patient interface 800. To this end, the exit lens 710 of the optics assembly 1001 has an eye-facing, concave surface, and a convex surface opposite the concave surface. The convex surface of the exit lens 710 corresponds to the incident surface or entry surface of the optic assembly 1001. The concave surface is configured to couple to a convex surface of a window 801 of the patient interface 800. In some embodiments, like shown in FIGS. 6A and 6B, the exit lens 710 is aspheric and has a meniscus form. In other configurations the exit lens may be a spherical lens. In one embodiment, the exit lens 710 is formed of fused silica and has a refractive index nₓ of 1.45.

The patient interface 800 optically and physically couples the eye 1 to the exit lens 710. The patient interface 800 immobilizes the eye relative to components of the integrated surgical system, creates a sterile barrier between the components and the patient, and provides optical access between the eye and the instrument. The patient interface 800 is a sterile, single use disposable device and it is coupled detachably to the eye 1 and to the exit lens 710. The patient interface 800 includes a window 801 having an eye-facing, concave surface and an objective-facing, convex surface opposite the concave surface. The window 801 thus has a meniscus form. The concave surface is configured to couple to the eye, either through a direct contact or through index matching material, liquid or gel, placed in between the concave surface and the eye 1. The window 801 may be formed of glass and has a refractive index my. In one embodiment, the window 801 is formed of fused silica and has a refractive index my of 1.45.

The window 801 is surrounded by a wall 803 of the patient interface 800 and an immobilization device, such as a suction ring 804. When the suction ring 804 is in contact with the eye 1, an annular cavity 805 is formed between the suction ring and the eye. When vacuum applied to the suction ring 804 and the cavity via a vacuum tube a vacuum pump (not shown in FIGS. 6A and 6B), vacuum forces between the eye and the suction ring attach the eye to the patient interface 800 during surgery. Removing the vacuum releases or detach the eye 1. The end of the patient interface 800 opposite the eye 1 includes an attachment interface 806 configured to attach to the non-rotatable distal portion 702b of the housing 702 of the focusing objective head 700 to thereby affix the position of the eye relative to the other components of the integrated surgical system 1000. The attachment interface 806 can work with mechanical, vacuum, magnetic or other principles and it is also detachable from the integrated surgical system.

Asymmetric Optics Assemblies

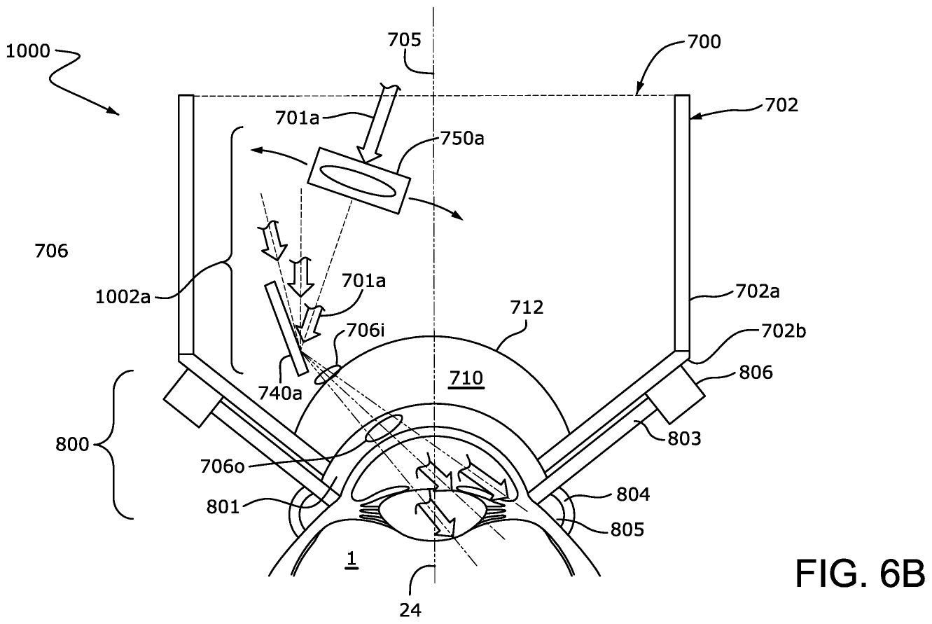

FIGS. 7A and 7B are schematic illustrations of an embodiment of a focusing objective head 700 of an integrated surgical system 1000 having an asymmetric optics assembly 1001 optically coupled with a first optical transmission subsystem 1002a and an optional second optical transmission subsystem 1002b, each of which are optically coupled with respective components (not shown) to respectively receive a first light beam 701a and a second light beam 701b. The optics assembly 1001 is mechanically coupled to a housing 702 of the focusing objective head 700. One or more components of the first optical transmission subsystem 1002a and the optional second optical transmission subsystem 1002b are also mechanically coupled to the housing 702. Thus, in this embodiment the optics assembly 1001, the first optical transmission subsystem 1002a and the second optical transmission subsystem 1002b (if present) may be rotated relative to the eye. While details of the mechanical coupling of the optics assembly 1001, the first optical transmission subsystem 1002a, and the second optical transmission subsystem 1002b to the housing 702 are not illustrated, various means or mechanisms may be used to secure these components to the interior of the housing at appropriate locations to maintain their respective optical couplings.

Continuing with the embodiment of FIGS. 7A and 7B, the optics assembly 1001 includes a prism 752 and an exit lens 710 having a reflecting surface 742. The first optical transmission subsystem 1002a is optically coupled to receive the first light beam 701a and to direct the first light beam incident a surface 712 of the exit lens 710 along a first input axis 706ᵢ. The surface 712 of the exit lens 710 to which the first light beam 701a is incident may be referred to herein a first incident surface of the optics assembly 1001 or a first entry surface of the optics assembly. The reflecting surface 742 of the exit lens 710 receives the first light beam 701a and reflects the first light beam into alignment with a first output axis 7060 of the optics assembly 1001. The first light beam 701a may be a laser beam, an OCT beam, a visual observation beam, dual aiming beams, or any other type of light beam or a combination thereof.

The optional second optical transmission subsystem 1002b is optically coupled to receive a second light beam 701b and to direct the second light beam incident an entry face 753 of the prism 752 along a second input axis 707ᵢ. The entry face 753 of the prism 752 may be referred to herein a second incident surface of the optics assembly 1001 or a second entry surface of the optics assembly. The prism 752 directs the second light beam 701b to an exit face 755 of the prism and into the exit lens 710, which in turn directs the second light beam into alignment with a second output axis 707o of the optics assembly 1001. The second light beam 701b may be a laser beam, an OCT beam, a visual observation beam, dual aiming beams, or any other type of light beam or a combination thereof.

Although not illustrated in FIGS. 7A and 7B, the first optical transmission subsystem 1002a may include either or each of a first focusing objective and a first alignment mechanism, and the second optical transmission subsystem 1002b may include either or each of a second focusing objective and a second alignment mechanism as described in the embodiments of FIGS. 6A and 6B. The first optical transmission subsystem 1002a and the second optical transmission subsystem 1002b may include other components, such as beam conditions and scanners 500 and beam combiners 600.

The optics assembly 1001 has an optical axis 705. The optics assembly 1001 is configured to couple to the eye through a patient interface 800 to align its optical axis 705 with the optical axis 24 of the eye. The optics assembly 1001 is optically coupled with the first optical transmission subsystem 1002a to receive the first light beam 701a along one of a plurality of first input axes 706ᵢ incident a first incident surface 712 of the optics assembly and to direct the light beam through the optics assembly to a corresponding one of a plurality of first output axes 706ₒ aligned with a corresponding one of the plurality of target volumes of ocular tissue in the eye.

With reference to FIG. 7B (wherein for clarity of illustration, the optional second optical transmission subsystem 1002b is not shown), in some configurations the first optical transmission subsystem 1002a comprises a means to direct the first light beam 701a into alignment with a select one of first input axes 706ᵢ of the optics assembly 1001. To this end, the first optical transmission subsystem 1002a may include a first alignment mechanism like the one described above for the embodiments of FIGS. 6A and 6B. The first alignment mechanism, e.g., actuated flip mirror, an actuated angular deflector, or fiber-optic cable, may be positioned and oriented relative to the first incident surface of the optics assembly 1001 and configured to be repositioned and/or reoriented relative to the incident surface to direct the first light beam 701a into alignment with the selected first input axis 706ᵢ of the optics assembly.

With continued reference to FIG. 7B, in other configurations one or more components of the first optical transmission subsystem 1002a may be configured to move relative to the optics assembly 1001 to align the first light beam 701a with a select one of first input axes 706ᵢ of the optics assembly. To this end, the first optical transmission subsystem 1002a may include a first focusing objective like the one described above for the embodiments of FIGS. 6A and 6B. The first focusing objective may be positioned and oriented relative to the incident surface 712 of the optics assembly and configured to be mechanically repositioned and/or reoriented relative to the incident surface to direct the first light beam 701a into the exit lens 710 along the selected first input axis 706ᵢ of the optics assembly.

In other configurations, the first optical transmission subsystem 1002a may include both of a first alignment mechanism and a first focusing objective. In the foregoing example embodiments of FIG. 7B, three different first input axes 706ᵢ of the optics assembly 1001 are illustrated. It is understood, however, that the number of first input axes 706ᵢ is not limited to three and may essentially comprise an almost infinite number of axes.

With reference to FIG. 7A, the optics assembly 1001 is optically coupled with the second optical transmission subsystem 1002b (if present) to receive the second light beam 701b along one of a plurality of second input axes 707ᵢ incident the second incident surface 753 of the optics assembly and to direct the second light beam to a corresponding one of a plurality of second output axes 707ₒ aligned with a corresponding one of the plurality of target volumes of ocular tissue in the eye. The second optical transmission subsystem 1002b may be configured the same as the first optical transmission subsystem 1002a as described above.

In the embodiment of FIGS. 7A and 7B, components of the first optical transmission subsystem 1002a and the second optical transmission subsystem 1002b (if present) are configured to rotate about the optical axis 705 of the optics assembly 1001 together with the optics assembly while the patient interface 800 remains fixed in place. To this end, the optics assembly 1001 is mechanically coupled to a rotatable housing 702 of the focusing objective head 700 together with one or more components of the first optical transmission subsystem 1002a. The components of the first optical transmission subsystem 1002a are arranged relative to other components (e.g., beam conditioners and scanners 500 and beam combiners 600) to maintain optical coupling with these components to receive the first light beam 701a regardless of the rotational position of the rotatable housing 702. Similarly, if a second optical transmission subsystem 1002b is present, one or more components of the first optical transmission subsystem may be mechanically coupled to the housing 702 and arranged relative to other components (e.g., beam conditioners and scanners 500 and beam combiners 600) to maintain optical coupling with these components to receive the second light beam 701b regardless of the rotational position of the housing 702. Rotating the housing 702 of the focusing objective head 700 as such, the optics assembly 1001, the first light beam 701a and the second light beam 701b rotate together relative to the fixed window 801 of the patient interface 800 and around the optical axis 705 of the optics assembly. This allows optical access to various tissue targets of the eye around the 360-degree circumference of the eye 1 by each of light beams 701a, 701b.

The optics assembly 1001 provides an interface to the eye through the patient interface 800. To this end, the exit lens 710 of the optics assembly 1001 has an eye-facing, concave surface, and a generally convex surface 712 opposite the concave surface. The prism 752 includes an input surface or entry face 753 and an output surface or exit face 755. The generally convex surface 712 of the exit lens 710 includes a modified surface 719 configured to couple to the exit face 755 of the prism 752. In one configuration the modified surface 719 is a flat surface having a geometry that matches the geometry of the exit face 755 of the prism 752. The geometry may be, for example, a rectangle. The exit lens 710 may thus be described as having a generally meniscus form with a modified surface 719. The exit lens 710 and the prism 752 may be formed of a solid material the same as or similar to the window 801 of the patient interface 800. In one embodiment, the exit lens 710 is formed of fused silica and has a refractive index nₓ of 1.45. The prism 752 may be formed of fused silica and has a refractive index n p that is the same as that of the exit lens 710. The patient interface 800 optically and physically couples the eye 1 to the exit lens 710. The patient interface 800 may be configured the same as described above with reference to FIGS. 6A and 6B.

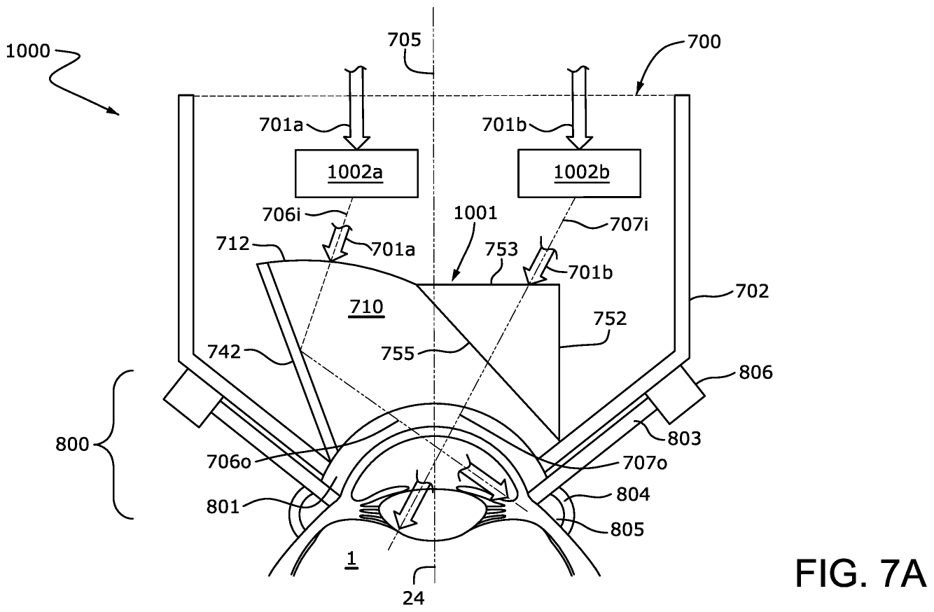

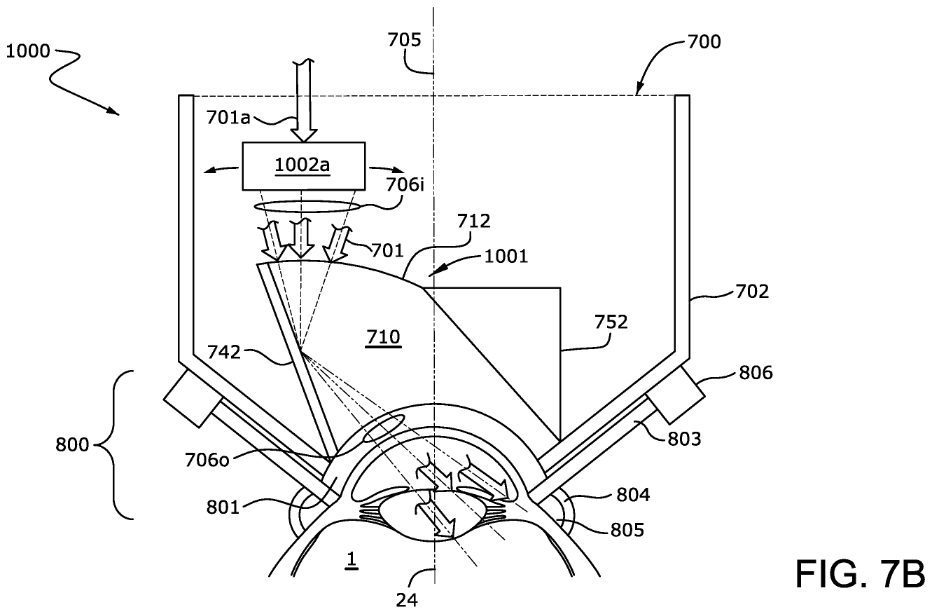

With reference to FIGS. 8A, 8B, 8C, 8D, and 8E, alternative configurations of the optics assembly 1001 are contemplated, each of which may be used in place of the optics assembly of FIGS. 7A and 7B. In each of FIGS. 8A, 8B, 8C, 8D, and 8E, the optics assembly 1001 includes an exit lens 710 that has an entry surface 712 and an extended side region with a reflecting surface 742. The entry surface 712 receives the first light beam 701a along a first input axis 706ᵢ incident the entry surface. The reflecting surface 742 receives the first light beam 701a and reflects the first light beam into alignment with a first output axis 706o of the optics assembly 1001.

In FIGS. 8A and 8B, the prisms 752 are similar to the prism of FIGS. 7A and 7B. These prisms 752 receive a second light beam 701b incident an entry face 753a, 753b of the prism along a second input axis 707ᵢ and direct the second light beam 701b to an exit face 755 of the prism and into the exit lens 710, which in turn directs the second light beam into alignment with a second output axis 707o of the optics assembly 1001.

In FIGS. 8C, 8D and 8E, each of the prisms 752 has one or more reflecting surfaces off which the second light beam 701b reflects into an exit lens 710, which in turn directs the second light beam into alignment with a second output axis 707o of the optics assembly 1001. In these configurations, the prisms 752 include: 1) an entry face 753, 2) one or more reflective facets 757, 759, and 3) an exit face 755. These configurations of prisms 752 are solutions that address two challenges: 1) the very tight space constraints within the focusing objective head 700, and 2) ensuring the second light beam 701b reaches the target volume 720.

With reference to the optical assembly of FIG. 8E, due to the curved top surface 712e of the exit lens 710e, without the prism 752e, the angle of incidence of the input light beam along the second input axis 707eᵢ would be high and the refraction angle steep. Considering the extremely limited space within the focusing objective head 700, it may be geometrically difficult to direct the second light beam 701b at an angle relative to curved top surface 712e of the exit lens 710e such that the second light beam would strike the target volume 720. To address this, the prism 752 of FIG. 8E has three faces: an entry face 753, a reflective surface 757 and an exit face 755. The prism 752 is designed and mechanically positioned so that the nominal second light beam 701b angle of incidence on the entry face 753 is normal, and the second light beam scanner (if any) has minor deviation (+/−10 degrees), which minimizes the scanned beams refraction and therefore aberrations. There are several other design measures implemented to reduce the refractive angle change. Firstly, the prism 752 is made from the same material as the exit lens 710. Secondly, a region of the top surface 712 is modified to have an angled flat face machined for prism placement and registration to bond the prism to the exit lens 710. The modified surface 719 includes an angled flat face that is parallel to the exit face 755 of the prism 752. As the exit face 755 of the prism 752 and modified surface 719 are parallel and the prism and exit lens 710 are of the same material, there is no refraction when the second light beam 701b traverses the prism-lens interface.

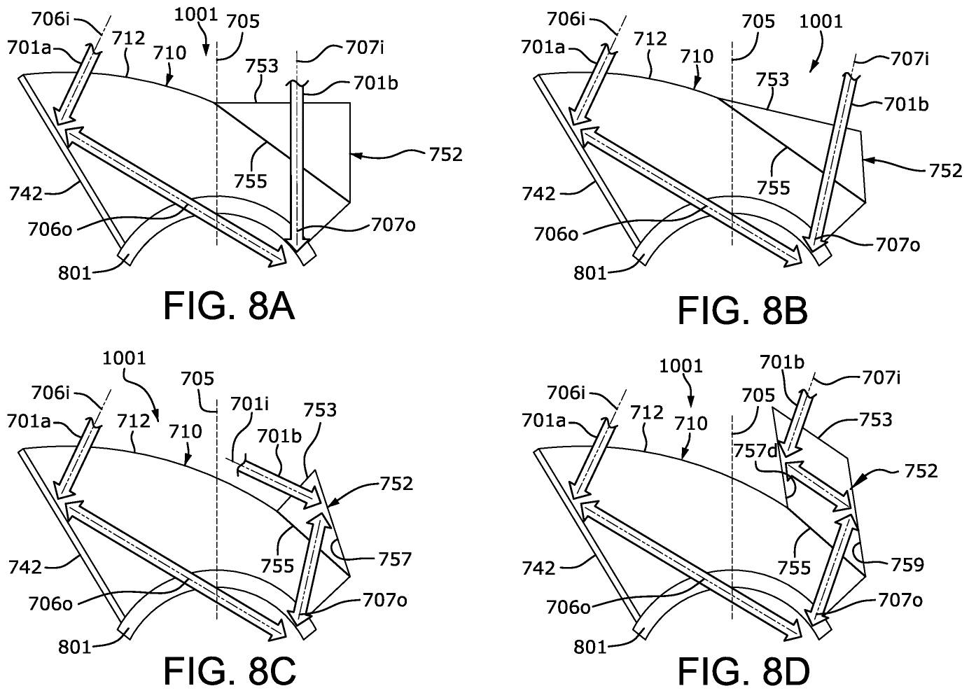

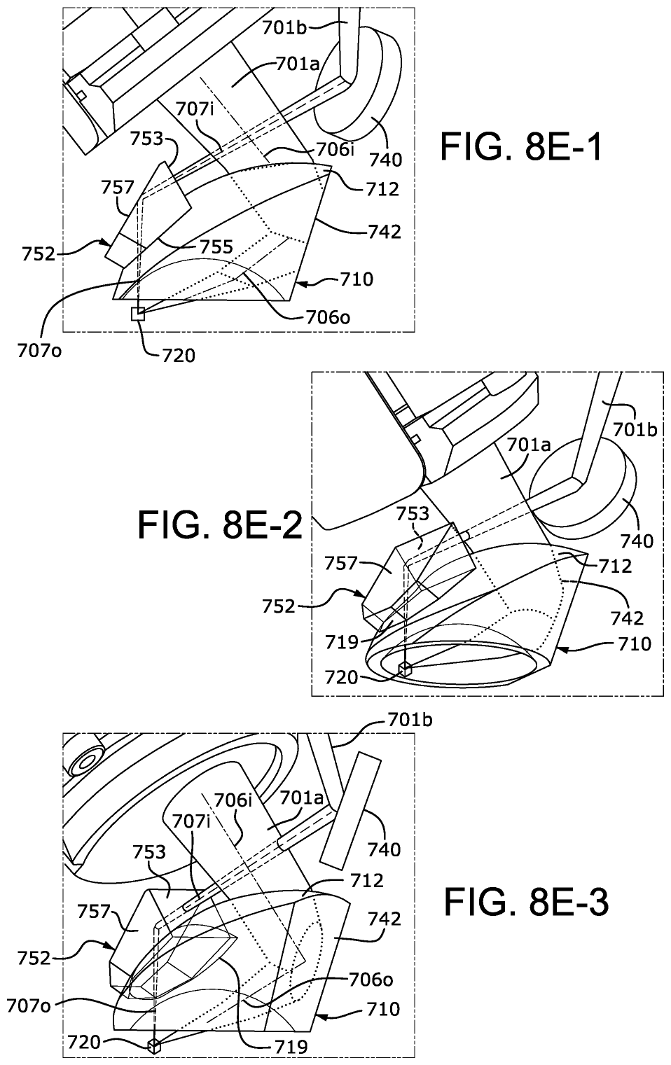

With reference to FIGS. 7A and 7B, the focusing objective head 700 and the patient interface 800 may be configured such that focusing objective head 700 is able to rotate within the additional component without any rotational torque being transferred to the patient interface 800 that is secured to the eye. To this end, an additional component may be included between the focusing objective head 700 and the patient interface 800. The additional component is fixed in place relative to the patient interface 800 but not the focusing objective head 700. FIGS. 9 and 10 are examples of such configurations.

FIG. 9 is a schematic illustration of a configuration where a housing 702 of a focusing objective head 700 is docked together with a patient interface 800 through an interface structure 810a that couples to the patient interface. This docking results in an indirect-contact coupling between the optics assembly 1001 of the focusing objective head 700 and the window 801 of the patient interface 800. The housing 702 of the focusing objective head 700 and the interface structure 810a are mechanically configured and coupled together to enable rotation of the optics assembly 1001 and the optical transmission subsystems 1002a, 1002b within the interface structure, relative to the patient interface 800, which is configured to fixedly couple to the eye and decouple therefrom. In this configuration, the optical transmission subsystems 1002a, 1002b can rotate about the optical axis 705 of the optics assembly 1001 without any rotational torque being transferred to the patient interface 800 that is secured to the eye. The attachment interface 806 of the patient interface 800 attaches to the non-rotating interface structure 810a. Thus, rotation of the focusing objective head 700 does not translate rotational toque to the patient interface coupled to the eye. In this configuration, the interface structure 810a includes a transparent window 811 through which light beams 701a, 701b pass into the window 801 of the patient interface 800.

FIG. 10 is a schematic illustration of other configurations where a housing 702 of the focusing objective head 700 is docked together with a patient interface 800 through an interface structure 810b that couples to the patient interface. This docking results in an indirect-contact coupling between the optics assembly 1001 of the focusing objective head 700 and the window 801 of the patient interface 800. Like the configuration of FIG. 9, the housing 702 of the focusing objective head 700 and the interface structure 810b are mechanically configured and coupled together to enable rotation of the optics assembly 1001 and the optical transmission subsystems 1002a, 1002b within the interface structure, relative to the patient interface 800, which is configured to fixedly couple to the eye and decouple therefrom. In this configuration, the optical transmission subsystems 1002a, 1002b can rotate about the optical axis 705 of the optics assembly 1001 without any rotational torque being transferred to the patient interface 800 that is secured to the eye. The attachment interface 806 of the patient interface 800 attaches to the non-rotating interface structure 810a. Thus, rotation of the focusing objective head 700 does not translate rotational toque to the patient interface coupled to the eye. In this configuration, the interface structure 810b includes an opening 814 through which through which light beams 701a, 701b pass into the window 801 of the patient interface 800. An index matching material, liquid, or gel 807 is placed on the convex surface of the window 801 to create a layer between the exit lens 710 and the window upon coupling of the components 800, 810b, 700.

Laser Treatment Overview

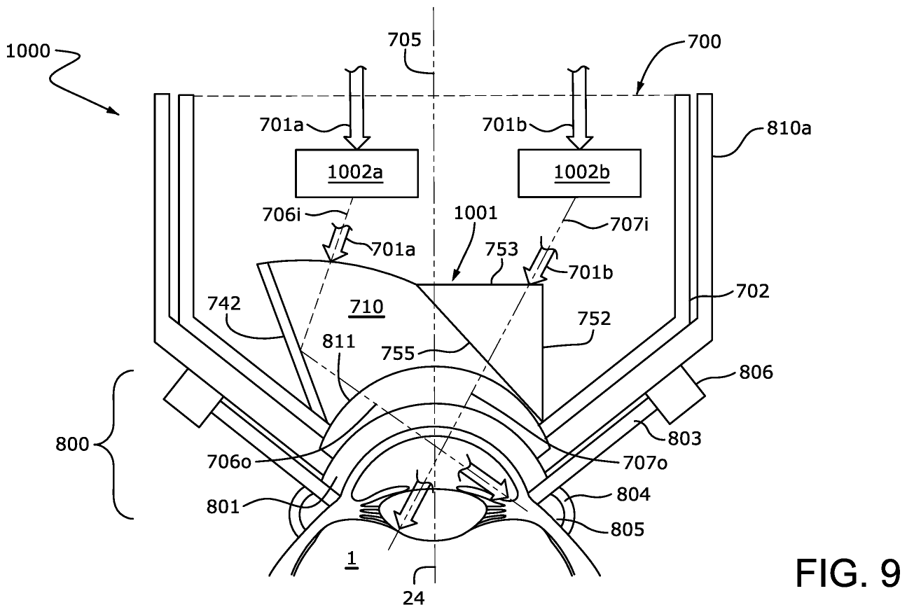

FIG. 11 is a three-dimensional schematic illustration of ocular tissue in the irido-corneal angle of the eye relevant to a surgical treatment for glaucoma. The ocular tissues may include the trabecular meshwork 12, the scleral spur 14, the Schlemm's canal 18, and the collector channels 19. The trabecular meshwork 12 has three layers, the uveal 15, the corneoscleral meshwork 16, and the juxtacanalicular tissue 17. A target volume 900 of ocular tissue of ocular tissue, also referred to as a surgical volume, comprises a portion of the trabecular meshwork 12.

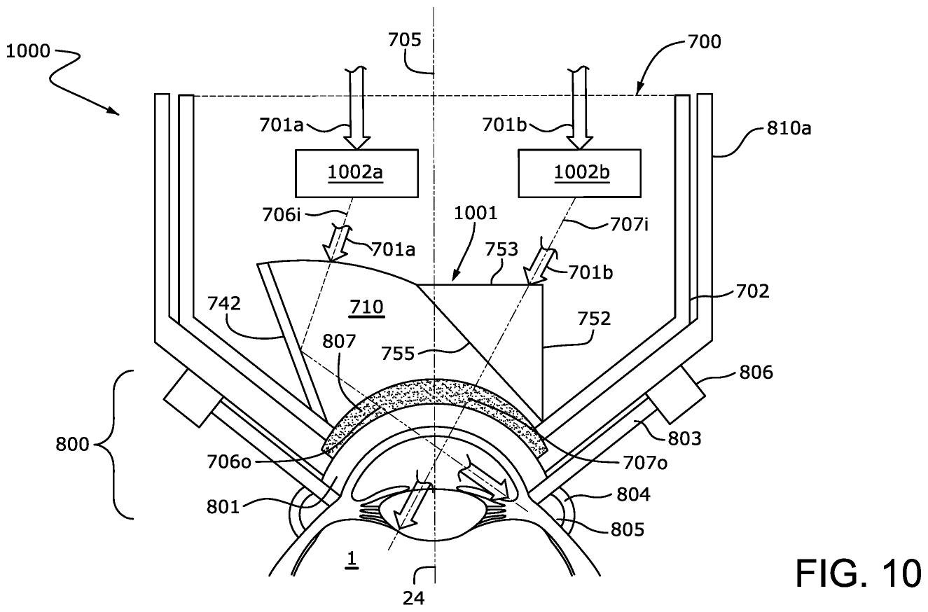

FIG. 12 includes a three-dimensional illustration of a treatment pattern P1 and a two-dimensional schematic illustration of the treatment pattern P1 overlaying the target volume 900 of ocular tissue of ocular tissue. The treatment pattern P1 is applied by the integrated surgical system 1000 to scan a laser beam 201 through the target volume 900 of ocular tissue of ocular tissue and photodisrupt the target volume of ocular tissue.

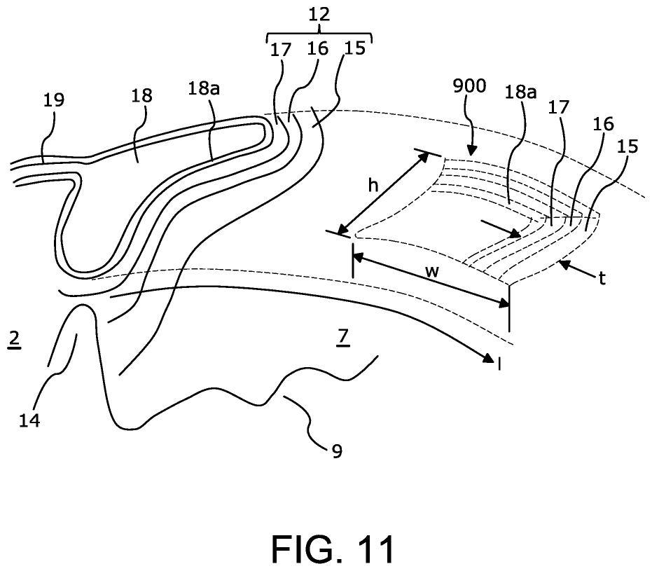

FIG. 13 is a three-dimensional schematic illustration of the ocular tissue in the irido-corneal angle of the eye including an opening 902 through the trabecular meshwork 12 that results from the application of the laser treatment pattern P1 of FIG. 12. The opening 902 may also be referred to as a channel, aperture, or void. The opening 902 provides an outflow pathway 40 through the trabecular meshwork 12 that reduces the flow resistance in the ocular tissue to increase aqueous flow from the anterior chamber 7 into the Schlemm's canal 18 and thereby reduce the intraocular pressure of the eye.

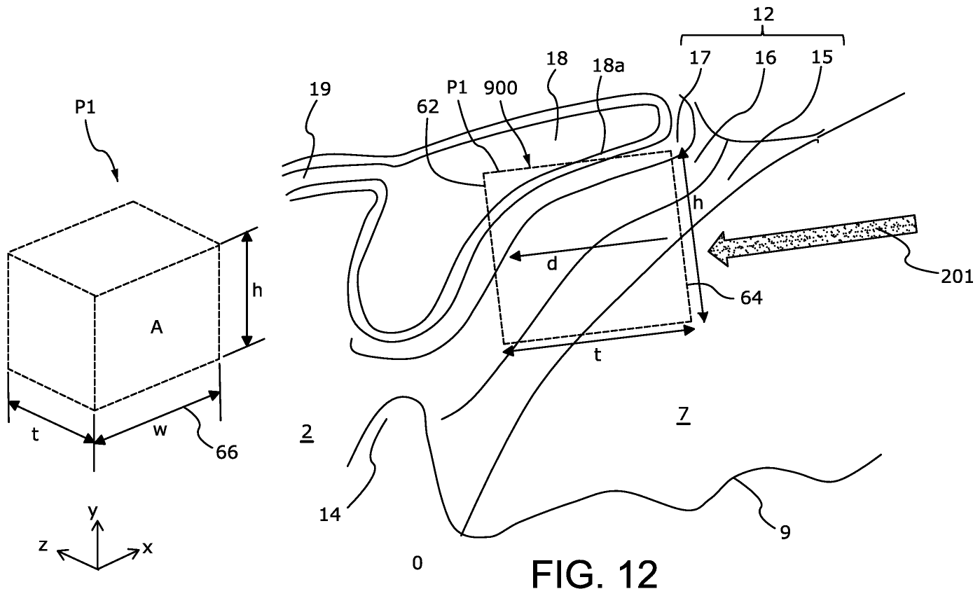

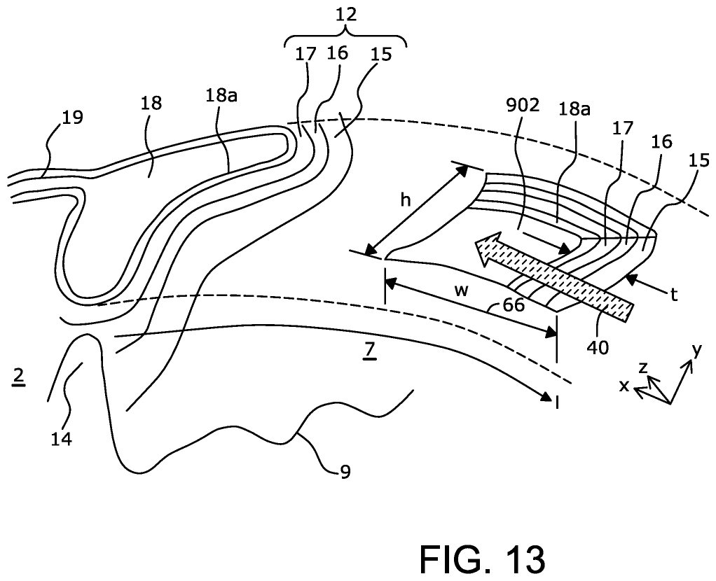

As shown in FIG. 12, and with additional reference to FIGS. 14A and 14B, a three-dimensional (3D) treatment pattern P1 may be in the form of a cubic structure that encompasses individual cells or spots arranged in regularly spaced rows, columns and sheets or layers. The treatment pattern P1 may be characterized by x, y, z dimensions, with x, y, z coordinates of the spots being assigned sequentially from neighbor to neighbor in the order of a column location (x coordinate), a row location (y coordinate), and a layer location (z coordinate). A treatment pattern P1 as such, defines a three-dimensional model of ocular tissue to be modified by a laser.

With reference to FIG. 12, a treatment pattern P1 is typically defined by a set of surgical parameters. The surgical parameters may include one or more of a treatment area A that represents a surface area or layer of ocular tissue through which a laser travels. The treatment area A is determined by the treatment height h and the lateral extent or circumferential extent or treatment width w. The width w may be defined in terms of a measure around the circumferential angle. For example, the width w may be defined in terms of an angle, e.g., 90 degrees, around the circumferential angle. A treatment thickness t represents the level to which the laser cuts into the ocular tissue from a distal extent 62 or border of the treatment volume at or near Schlemm's canal 18 to a proximal extent 64 or border at or near the surface of the trabecular meshwork 12. Thus, a laser applied in accordance with a treatment pattern may affect or produce a surgical volume that resembles the three-dimensional model of the treatment pattern.

Additional surgical parameters may define the placement of the target volume 900 of ocular tissue of ocular tissue and the resulting opening 902 within the eye. For example, with reference to FIGS. 11-13, placement parameters may include one or more of a location l (shown in FIGS. 11 and 13) that represents where the treatment is to occur around the circumferential angle of the eye, and a treatment depth d (shown in FIG. 12) that represents a position of the three-dimensional model of ocular tissue relative to a reference eye structure. In the following, the treatment depth d is shown and described relative to the region where the anterior chamber 7 meets the trabecular meshwork 12. Together, the treatment pattern h, w, and t, and the placement parameters l and d define a treatment plan.

With reference to FIGS. 14A and 14B, a 3D treatment pattern P1 may be defined by a number of 2D treatment layers 1402 or treatment planes that are stacked to form a 3D treatment pattern characterized by a width w, height h, and depth or thickness t. Each individual treatment layer 1402 is in turn characterized by a pattern height h (equal to the height h of the 3D treatment pattern P1) and a pattern width w (equal to the width w of the 3D treatment pattern P1) and comprises an array of spots 1404 spaced apart to establish or fit within the height and width. The pattern width w corresponds to a distance along the circumference of the corneal angle parallel to the trabecular meshwork. This direction is also known as the circumferential direction. The pattern height h corresponds to a distance transverse to the circumference of the corneal angle perpendicular to the trabecular meshwork. This direction is also known as the azimuthal direction.

Each spot 1404 in the treatment pattern P1 corresponds to a site within a target volume 900 of ocular tissue where optical energy is applied at a laser focus to create a micro-photodisruption site. With reference to FIG. 14B, each spot 1404 in a treatment layer 1402 is separated from a neighboring spot by programmable distances called spot separation (Spot Sep 1406) and a line separation (Line Sep 1408). A treatment layer 1402 is completed with the programmed pattern width w 1410 and pattern height h 1412 is achieved. Each layer 1402 in the 3D treatment pattern P1 is separated from a neighboring layer by a layer separation (Layer Sep).

A treatment pattern P1 may be defined by a set of programmable parameters, such as shown in Table 1.

TABLE 1

| | | |
|---|---|---|
| Parameter | Minimum | Maximum |

| | | | | |
|---|---|---|---|---|
| width w | 10 | μm | 2000 | μm |
| height h | 10 | μm | 2000 | μm |
| depth/thickness t | 10 | μm | 4000 | μm |
| Spot Sep | 2 | μm | 40 | μm |
| Line Sep | 2 | μm | 40 | μm |
| Layer Sep | 2 | μm | 200 | μm |
| pulse energy | 0 | μJ | 35 | μJ |

Other, non-rectangular and more irregular treatment patterns can also be programmed and created in the tissue. These irregular patterns can still be decomposed to spots, lines, and layers and their extent characterized by width, height, and depth. Examples of irregular treatment patterns are described in U.S. Patent Application Publication No. 2021/0307964, entitled Method, System, and Apparatus for Generating Three-Dimensional Treatment Patterns for Laser Surgery of Glaucoma, the disclosure of which is hereby incorporated by reference.

With reference to FIGS. 12 and 13, during laser treatment, a surgical laser beam 201 may be scanned through the target volume 900 of ocular tissue in accordance with the treatment pattern P1 to form an opening 902 that extends from the anterior chamber 7, through each of the uveal 15, the corneoscleral meshwork 16, the juxtacanalicular tissue 17 of the trabecular meshwork 12, and the inner wall 18a of the Schlemm's canal 18. The opening 902 resulting from laser application of the treatment pattern P1 resembles the treatment pattern and is characterized by an area A and thickness t similar to those of the treatment pattern. The thickness t of the resulting opening 902 extends from the anterior chamber 7 and through the inner wall 18a of the Schlemm's canal 18, while the area A defines the cross-section size of the opening 902.

With reference to FIGS. 14A and 14B, in some laser treatments, each treatment layer 1402 is individually created by scanning the laser focus in two dimensions, e.g., width and height, or x and y, to the various spots 1404 defining the layer, while the focus is fixed at the third dimension, e.g., depth or z. The laser scanning in two dimensions may be in the form of a raster scan, a spiral scan or a concentric circular scan. Once a treatment layer 1402 is created, the focus is moved in the depth or z direction and the next treatment layer in the stack is created. This process is repeated until all treatment layers 1402 in the 3D treatment pattern P1 are created. Details on this type of scanning are disclosed in U.S. Patent Application Publication No. 2021/0220176, the entire disclosure of which is hereby incorporated by reference.

In another treatment, instead of creating a treatment pattern P1 one treatment layer 1402 at a time, the focus of the laser beam 201 is scanned in three dimensions. For example, while the laser focus is being moved transversely through a height and/or width, e.g., in the x and/or y direction, the laser focus is also oscillated back and forth axially through a depth, e.g., in the z direction. The treatment pattern P1 characterized by such scanning of the laser focus may be referred to as a “clearing pattern.” Oscillation of the laser focus through the depth in the z direction occurs simultaneous with transverse movement of the laser focus in the x and y directions. An example of scanning a laser in accordance with a clearing pattern is also disclosed in U.S. Patent Application Publication No. 2021/0220176.

Laser Treatment Based on Tissue Pigmentation

FIG. 15 is a flowchart of a method of treating a target volume of ocular tissue of an eye based on pigmentation of the tissue. The method of FIG. 15, which may be performed by the integrated surgical system 1000 of FIGS. 4 through 7B, begins at a point in a surgical procedure where access to a target volume 900 of ocular tissue has been obtained. To this end, and for purposes of example only, access to a target volume 900 of ocular tissue corresponding to the trabecular meshwork in the irido-corneal angle 13 of the eye may be obtained by an integrated surgical system 1000 as shown and described above with reference to FIGS. 5A through 7B. The method of FIG. 15 also begins at a point in a surgical procedure where a treatment pattern P1 has been established for the procedure. To this end, and again for purposes of example only, a treatment pattern P1 for laser treatment of the trabecular meshwork may be established by the integrated surgical system 1000 as described above in the Laser Treatment Overview section of this disclosure with reference to FIGS. 11 through 14B.

Continuing with FIG. 15, at block 1502 an energy parameter is determined based on pigmentation of the target volume 900 of ocular tissue. Generally, the energy parameter corresponds to a minimum energy level that ensures photodisruption of ocular tissue by a laser 201, such as a femtosecond laser, as the laser is scanned through the target volume 900 of ocular tissue. An energy parameter can be determined based on pigmentation of the target volume 900 of ocular tissue as captured in a color image.

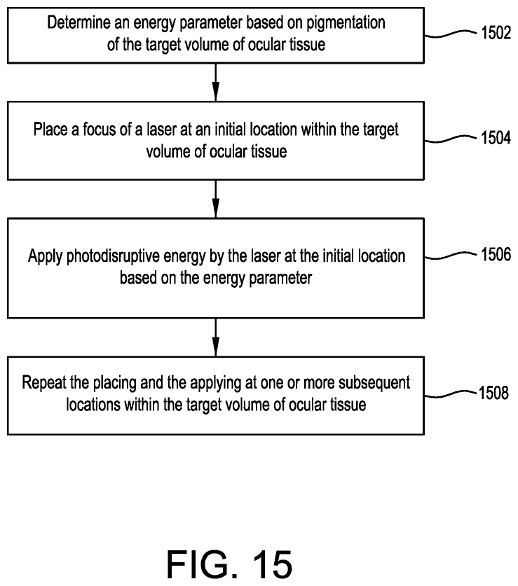

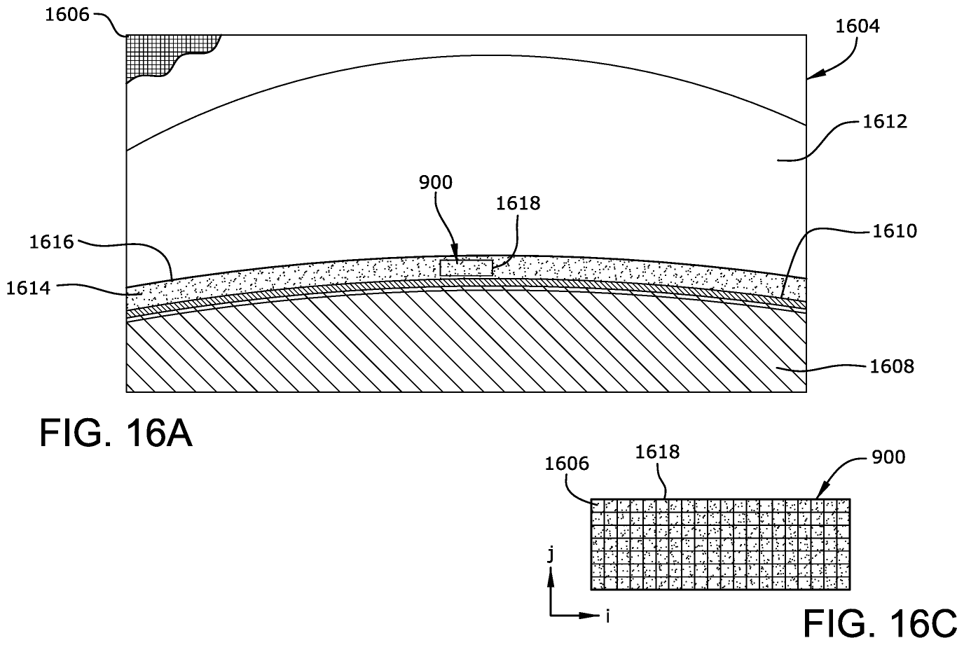

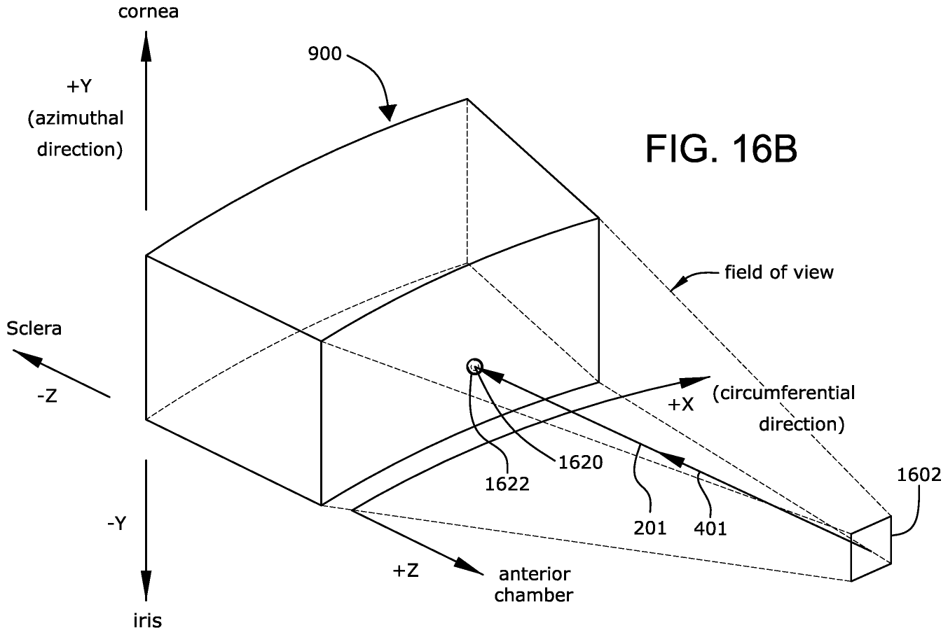

With reference to FIGS. 4 and 16A, a color image 1604 of the irido-corneal angle 13 of the eye that includes the target volume 900 of ocular tissue is captured by a visual observation apparatus 400 of the integrated surgical system 1000. The color image 1604 is a digital image captured by an image sensor and comprises an array of pixels 1606 each identifiable by an i, j coordinates, where i is the horizontal axis location or column location of a pixel, and j is the vertical axis location or row location of the pixel.

The visual observation apparatus 400 may include a gonioscope camera having a field of view that captures various anatomical features of the irido-corneal angle 13. For example, the color image 1604 may include the ciliary body band and iris 1608, scleral spur 1610, the cornea 1612, the trabecular meshwork 1614 and Schwalbe's line 1616. The color image 1604 may be presented on a display of the control system 100. The control system 100 may be configured to display a surgical overlay mark 1618 over the color image 1604 that identifies the boundaries of the target volume 900. In the example color image 1604 of FIG. 16A, the target volume 900 is within a region of the trabecular meshwork 1614.

With reference to FIGS. 4, 16A, and 16B, to capture the color image 1604 a visual observation beam 401 output by the visual observation apparatus 400 of the integrated surgical system 1000 is directed along a beam path to the target volume 900 ocular tissue. In some embodiments, the beam path is the same beam path along which a laser beam 201 will be directed for purposes of laser treatment of the target volume 900 ocular tissue. In some embodiments, the beam path of the visual observation beam 401 may be a different beam path than the laser beam path. In either case, an image sensor 1602 included in the camera, e.g., a gonioscope camera, of the visual observation apparatus 400 captures the color image. In the example of FIGS. 16A and 16B, the color image 1604 captured by the image sensor 1602 includes a surface or region of the target volume 900 of ocular tissue facing the anterior chamber. Note that in FIG. 16B the field of view of the image sensor 1602 is limited to the target volume 900 of ocular tissue, with the surrounding anatomical features not shown for clarity of illustration.

With reference to FIGS. 16A and 16C, as previously mentioned the color image 1604 is a digital image and comprises an array of pixels 1606 each identifiable by an i, j coordinates, where i is the horizontal axis location or column location of a pixel, and j is the vertical axis location or row location of the pixel. With additional reference to FIG. 16B, the horizontal axis of the color image 1604 is generally aligned relative to the circumferential direction of the laser scan, and the vertical axis the color image 1604 is generally aligned relative to the azimuthal direction of the laser scan.

The color image 1604 obtained by the image sensor 1602 is processed by an algorithm resident in a processor of the control system 100 to determine a composite pigmentation metric for the target volume 900 of ocular tissue. The control system 100 then determines the energy parameter based on the composite pigmentation metric.

Regarding the composite pigmentation metric, it is generally determined based on comparisons of colors present in the color image 1604 of the target volume 900 of ocular tissue and a reference color. More specifically, and with reference to FIG. 16C, an individual pigmentation metric is determined for each pixel 1606 in the color image 1604 of the target volume 900 of ocular tissue based on the color of the pixel and the reference color; and the composite pigmentation metric is determined based on the plurality of individual pigmentation metrics. In some embodiments, the reference color and the pixel colors are characterized by a red value, a green value, and a blue value, collectively a RGB value. In some embodiments, the reference color corresponds to the darkest color in the color image 1604 of the target volume 900 of ocular tissue.

In some embodiments, individual pigmentation metrics are determined using vector calculus. To this end, the control system 100 is configured to calculate an individual pigmentation metric as an angle θ_{I} between a reference color vector corresponding to the reference color and a pixel color vector corresponding to the color of the pixel 1606. The individual pigmentation metric θ_{I} provides a measure of deviation between the reference color and the color of the pixel 1606.

The angle θ_{I} may be calculated based on the equation:

$$θ ( i , j ) = {cos}^{- 1} [ \frac{A \cdot B ( i , j )}{\vert A \vert \vert B \vert} ] \qquad ( Eq . 1 )$$

where:
A=the reference color vector; and
B=the pixel color vector for the pixel 1606 at column, row coordinate location i, j in the color image 1604 of the target volume 900 of ocular tissue.

In an example, the methodology determines how the color of a target volume 900 of ocular tissue corresponding to trabecular meshwork 12 compares to (or deviates from) the darkest pigmentation. Each pixel 1606 in a color image 1604 of the target volume 900 of ocular tissue has five values associated with it: two pixel position coordinates corresponding to the i (horizontal) and j (vertical) location of the pixel along the axes of the image sensor, and three color values corresponding to the primary color values (red, green, and blue) of the pixel. With reference to FIGS. 12 and 13, for the color image 1604 orientation shown in FIGS. 16A and 16C, the horizontal (x) and vertical (y) image sensor axes are aligned with the circumferential and azimuthal directions of the target volume 900 of ocular tissue shown in FIG. 16B.

For an 8-bit image, each red, green, and blue value ranges from 0 to 255. In this 8-bit scheme, (0, 0, 0) is black and (255, 255, 255) is white. In an example, if the darkest color of the target volume 900 of ocular tissue is defined to be a dark brown (165, 42, 42) and is used as the reference color vector A, then the angle θ_{I} (the individual pigmentation metric) between this reference color and any RGB vector B, is calculated at each i, j pixel 1606 of the color image 1604 of the target volume 900 of ocular tissue.

In an example calculation of angle θ_{I}, where A=(165, 42, 42) and B=(165, 42, 42), Equation 1 is processed by the control system 100 to determine a value 0 corresponding to angle θ_{I}. In this case, the color of the pixel 1606 (and thus the color of ocular tissue corresponding to that pixel) is exactly equal to the dark brown. In another example calculation of angle θ_{I}, where A=(165, 42, 42) and B=(75, 100, 100), Equation 1 is processed by the control system 100 to determine a value 42.26 corresponding to angle θ_{I}. In this case, the color of the pixel 1606 (and thus the color of ocular tissue corresponding to that pixel) deviates from dark brown. Generalizing the foregoing examples, where the reference color vector A is dark brown (165, 42, 42), the smaller the individual pigmentation metric θ_{I} is, the closer the color of the ocular tissue corresponding to that metric is to the reference color. Conversely the larger the composite pigmentation metric θ_{C} is, the further the color of the ocular tissue corresponding to that metric is from the reference color. In some embodiments, the composite pigmentation metric may be the average of the plurality of individual pigmentation metrics Oi. To this end, the control system 100 is configured to determine a composite pigmentation metric θ_{C} from the plurality of individual pigmentation metrics Oi by calculating an average of the plurality of individual pigmentation metrics based on the following equation:

$${θ}_{C} = \frac{1}{n} {{\sum }_{i}}_{j} θ ( i , j )$$

where:
n=the total number of pixels 1606 in the color image 1604 for which an individual pigmentation metric θ_{I} was determined

Continuing with the example above, where the reference color vector A is dark brown (165, 42, 42), the smaller the composite pigmentation metric θ_{C} the closer the color of the target volume 900 of ocular tissue is to the reference color, dark brown color. Conversely the larger the composite pigmentation metric θ_{C} is, the further the color of the trabecular meshwork target volume 900 of ocular tissue is from the reference color.

Regarding the determining of the energy parameter based on pigmentation, the control system 100 is configured to assign an energy parameter to be used for purposes of laser treatment of the target volume 900 of ocular tissue such that a composite pigmentation metric representing a pigment in a dark range is assigned a lower energy parameter, while a composite pigmentation metric representing a pigment in a light range is assigned a higher energy parameter. The fundamental concept implemented by the control system 100 in this regard is that the darker the target volume 900 of ocular tissue, the more pigment is present in the tissue. More pigment means more energy is absorbed by the tissue during photodisruption. More energy absorption means less energy is required to photodisrupt the tissue. Conversely, the lighter the target volume 900 of ocular tissue, the less pigment is present in the tissue. Less pigment means less energy is absorbed by the tissue during photodisruption. Less energy absorption means more energy is required to photodisrupt the tissue.

The control system 100 may assign an energy parameter to be used for purposes of laser treatment of the target volume 900 of ocular tissue based on a comparison between the composite pigmentation metric θ_{C} and a plurality of different pigmentation metrics, each having an associated energy parameter. The energy parameter may correspond to an energy level in the range of the approximately 0.8 to 1.0 μJ/cm² and is in the breakdown threshold range for ocular tissue. To this end, different composite pigmentation metric θ_{C} and associated energy parameters may be stored in a look up table. The look up table may be derived from clinical data across a population of patients with different pigmentation shades and corresponding required energy levels for tissue breakdown for their respective pigmentation shades. From this data set, an empirical relationship may be determined, where energy is proportional to pigmentation metric. The control system 100 compares the composite pigmentation metric θ_{C} of the target volume 900 of ocular tissue to the plurality of different pigmentation metrics and identifies which of the plurality of different pigmentation metrics is closest to the composite pigmentation metric. The control system 100 selects the energy parameter associated with of the identified pigmentation metric as the energy parameter to be used for purposes of laser treatment of the target volume 900 of ocular tissue.

In an alternative approach, instead of using RGB to characterize the pixel colors and the reference color, three layers: a luminosity or brightness layer and two chromaticity layers, where the color falls on a red-green axis and where it falls on a separate, independent blue-yellow axis are used. This is known as the L*a*b color space. The advantage of this approach is that the algorithm is less sensitive to the overall brightness of the image, or variations in brightness throughout an image for the same object (e.g., one side of the trabecular meshwork is brighter than the other side). In this approach, a look up table may assign an energy value to each L*a*b color space.

Returning to FIG. 15, and with continued reference to FIG. 16A, at block 1504 a focus 1620 of a laser 201 is placed at an initial location 1622 within the target volume 900 of ocular tissue.

At block 1506, photodisruptive energy is applied by the laser 201 at the initial location 1622 based on the energy parameter determined in block 1502.

At block 1508, the placing of the laser focus 1620 and the applying of photodisruptive energy based on the energy parameter is repeated at one or more subsequent locations within the target volume 900 of ocular tissue. This process of placing and applying may be repeated until the focus 1620 of the laser 201 has scanned through the target volume 900 of ocular tissue in accordance with the treatment pattern P1.

With reference to FIGS. 16A-16C, while the foregoing describes determining an energy parameter for photodisrupting a target volume 900 of ocular tissue based on numerical analyses (e.g., vector calculus) of tissue pigmentation, other methods may be used. In one alternative approach, clustering may be used to separate image pixels 1606 by value into clusters within a color space. In this approach, a color image 1604 of the target volume 900 of ocular tissue is obtained and applied to a clustering algorithm, such as a k-means clustering algorithm, which is configured to partition the image into “chunks of space”, or clusters of pixels 1606, wherein each pixel in a cluster has a similar color profile. The discretization resolution of the clustering algorithm may be determined by user parameters, such as number of clusters or color value, e.g., RGB value, limits. In this approach, a look up table may assign an energy value to each cluster, and thus to each pixel in the cluster. The energy parameter is determined based on the cluster that includes a pixel 1606 associated with the location of the laser focus 1620 within the target volume 900 of ocular tissue. A pixel 1606 of an image may be associated with the location of the laser focus 1620, for example, when the XY location of the pixel is at are near the XY location of the laser focus. As the laser focus 1620 moves to a different location 1622 within the target volume 900 of ocular tissue during the scanning process, the energy parameter may be adjusted based on the pixel associated with the different location of the laser focus 1620. In this case, the look up table may map XY laser scanning coordinates to XY image pixel coordinates, which in turn are mapped to clusters, which in turn are mapped to energy parameters.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of photodisrupting a target volume of ocular tissue of an eye with a laser, the method comprising:
determining an energy parameter based on pigmentation of the target volume of ocular tissue;
placing a focus of a laser at an initial location within the target volume of ocular tissue; and
applying photodisruptive energy by the laser at the initial location based on the energy parameter.

2. The method of claim 1, further comprising repeating the placing and the applying at one or more subsequent locations within the target volume of ocular tissue.

3. The method of claim 2, further comprising repeating the placing and the applying until the focus of the laser has scanned through the target volume of ocular tissue.

4. The method of claim 2, wherein the energy parameter corresponds to a minimum energy level that ensures photodisruption at the initial location and the one or more subsequent locations.

5. The method of claim 1, wherein determining an energy parameter based on pigmentation of the target volume of ocular tissue comprises:
obtaining, with an image sensor, a color image of the target volume of ocular tissue;
determining a composite pigmentation metric for the target volume of ocular tissue based on comparisons of colors present in the color image and a reference color; and
determining the energy parameter based on the composite pigmentation metric.

6. The method of claim 5, wherein the color image comprises an array of pixels and each of the colors present in the color image corresponds to a color of a pixel, and determining a composite pigmentation metric for the target volume of ocular tissue comprises:
for each of a plurality of pixels, determining an individual pigmentation metric based on the color of the pixel and the reference color; and
determining the composite pigmentation metric based on the plurality of individual pigmentation metrics.

7. The method of claim 6, wherein determining the composite pigmentation metric based on the plurality of individual pigmentation metrics comprises calculating an average of the plurality of individual pigmentation metrics.

8. The method of claim 6, wherein determining an individual pigmentation metric based on the color of the pixel and the reference color comprises:
calculating an angle θ_{I} between a reference color vector corresponding to the reference color and a pixel color vector corresponding to the color of the pixel.

9. The method of claim 8, wherein the array of pixels comprises a number i of columns and a number j of rows, and the angle θ_{I} is calculated based on:
$$θ ( i , j ) = {cos}^{- 1} [ \frac{A \cdot B ( i , j )}{\vert A \vert \vert B \vert} ]$$
where:
A=the reference color vector; and
B=the pixel color vector for the pixel at coordinate location i, j in the color image.

10. The method of claim 6, wherein the reference color corresponds to the darkest color in the target volume.

11. The method of claim 6, wherein determining the composite pigmentation metric based on the plurality of individual pigmentation metrics comprises calculating an average of the plurality of individual pigmentation metrics based on:
$${θ}_{C} = \frac{1}{n} {{\sum }_{i}}_{j} θ ( i , j )$$
where:
n=total number of pixels in the plurality of pixels.

12. The method of claim 5, wherein determining the energy parameter based on pigmentation comprises:
comparing the composite pigmentation metric to a plurality of different pigmentation metrics, each having an associated energy parameter; and
identifying which of the plurality of different pigmentation metrics the composite pigmentation metric is closest to and selecting the associated energy parameter of identified pigmentation metric as the energy parameter.

13. The method of claim 1, wherein the target volume of ocular tissue is at a first location around a circumferential angle of the eye, and the method further comprises:
repeating the determining, the placing, and the applying for a second volume of ocular tissue at a second location around the circumferential angle of the eye different from the first location.

14. The method of claim 1, wherein the target volume of ocular tissue is in one of an irido-corneal angle, a cornea, a crystalline lens, a posterior capsule of the lens, an anterior capsule of the lens, a vitreous humor, and a retina.

15. The method of claim 1, wherein the target volume of ocular tissue is in a trabecular meshwork of the eye.

16. The method of claim 1, wherein determining an energy parameter based on pigmentation of the target volume of ocular tissue comprises:
obtaining, with an image sensor, a color image of the target volume of ocular tissue;
applying the color image to a clustering algorithm configured to partition the color image into a plurality of clusters of pixels, wherein each pixel in a cluster has a similar color profile; and
determining the energy parameter based on the cluster that includes a pixel associated with the initial location.

17. An integrated surgical system for photodisrupting a target volume of ocular tissue of an eye with a laser, the integrated surgical system comprising:
a laser source configured to output a laser beam;
a visual observation apparatus configured to output a visual observation beam and to capture color images;
a plurality of components optically coupled to receive the laser beam and the visual observation beam and configured to one or more of focus, scan, and direct the laser beam and the visual observation beam;
an optics assembly configured to couple to the eye and optically coupled to receive the laser beam and the visual observation beam and to direct the laser beam and the visual observation beam to the target volume of ocular tissue; and
a control system coupled to the laser source, the visual observation apparatus, and one or more of the plurality of components, and configured to:
determine an energy parameter based on pigmentation of the target volume of ocular tissue,
control one or more of the plurality of components to place a focus of a laser beam from the laser source at an initial location within the target volume of ocular tissue; and
control the laser source to apply photodisruptive energy by the laser beam at the initial location based on the energy parameter.

18. The integrated surgical system of claim 17, wherein the control system is configured to repeat the placing and the applying at one or more subsequent locations within the target volume of ocular tissue.

19. The integrated surgical system of claim 18, wherein the control system is configured to repeat the placing and the applying until the focus of the laser has scanned through the target volume of ocular tissue.

20. The integrated surgical system of claim 18, wherein the energy parameter corresponds to a minimum energy level that ensures photodisruption at the initial location and the one or more subsequent locations.

21. The integrated surgical system of claim 17, wherein the control system determines an energy parameter based on pigmentation of the target volume of ocular tissue by being further configured to:
obtain a color image of the target volume of ocular tissue;
determine a composite pigmentation metric for the target volume of ocular tissue based on comparisons of colors present in the color image and a reference color; and
determine the energy parameter based on the composite pigmentation metric.

22. The integrated surgical system of claim 21, wherein the color image comprises an array of pixels and each of the colors present in the color image corresponds to a color of a pixel, and the control system determines a composite pigmentation metric for the target volume of ocular tissue by being further configured to:
for each of a plurality of pixels, determine an individual pigmentation metric based on the color of the pixel and the reference color; and
determine the composite pigmentation metric based on the plurality of individual pigmentation metrics.

23. The integrated surgical system of claim 22, wherein the control system determines the composite pigmentation metric based on the plurality of individual pigmentation metrics by being further configured to calculate an average of the plurality of individual pigmentation metrics.

24. The integrated surgical system of claim 22, wherein the control system determines an individual pigmentation metric based on the color of the pixel and the reference color by being further configured to:
calculate an angle θ_{I} between a reference color vector corresponding to the reference color and a pixel color vector corresponding to the color of the pixel.

25. The integrated surgical system of claim 24, wherein the array of pixels comprises a number i of columns and a number j of rows, and the angle θ_{I} is calculated based on:
$$θ ( i , j ) = {cos}^{- 1} [ \frac{A \cdot B ( i , j )}{\vert A \vert \vert B \vert} ]$$
where:
A=the reference color vector; and
B=the pixel color vector for the pixel at coordinate location i, j in the color image.

26. The integrated surgical system of claim 22, wherein the control system determines the composite pigmentation metric based on the plurality of individual pigmentation metrics by being further configured to calculate an average of the plurality of individual pigmentation metrics based on:
$${θ}_{C} = \frac{1}{n} {{\sum }_{i}}_{j} θ ( i , j )$$
where:
n=total number of pixels in the plurality of pixels.

27. The integrated surgical system of claim 21, wherein the control system determines the energy parameter based on pigmentation by being further configured to:
compare the composite pigmentation metric to a plurality of different pigmentation metrics, each having an associated energy parameter; and
identify which of the plurality of different pigmentation metrics the composite pigmentation metric is closest to and selecting the associated energy parameter of identified pigmentation metric as the energy parameter.

28. The integrated surgical system of claim 17, wherein the control system determines the energy parameter based on pigmentation of the target volume by being further configured to:
obtain, with an image sensor, a color image of the target volume of ocular tissue;
apply the color image to a clustering algorithm configured to partition the color image into a plurality of clusters of pixels, wherein each pixel in a cluster has a similar color profile; and
determine the energy parameter based on the cluster that includes a pixel associated with the initial location.

29. A control system coupled to a laser source configured to output a laser beam, a visual observation apparatus configured to output a visual observation beam and to capture color images, and one or more of a plurality of components coupled to receive the laser beam and the visual observation beam and configured to one or more of focus, scan, and direct the laser beam and the visual observation beam, the control system configured to:
determine an energy parameter based on pigmentation of a target volume of ocular tissue,
control one or more of the plurality of components to place a focus of a laser beam from the laser source at an initial location within the target volume of ocular tissue; and
control the laser source to apply photodisruptive energy by the laser beam at the initial location based on the energy parameter.

30. The control system of claim 29, wherein the control system determines an energy parameter based on pigmentation of the target volume of ocular tissue by being configured to:
determine a composite pigmentation metric for the target volume of ocular tissue based on comparisons of colors present in a color image captured by the visual observation apparatus and a reference color; and
determine the energy parameter based on the composite pigmentation metric.

31. The control system of claim 30, wherein the color image comprises an array of pixels and each of the colors present in the color image corresponds to a color of a pixel, and the control system determines a composite pigmentation metric for the target volume of ocular tissue by being further configured to:
for each of a plurality of pixels, determine an individual pigmentation metric based on the color of the pixel and the reference color; and
determine the composite pigmentation metric based on the plurality of individual pigmentation metrics.

32. The control system of claim 29, wherein the control system determines an energy parameter based on pigmentation of the target volume of ocular tissue by being configured to:
apply a color image captured by the visual observation apparatus to a clustering algorithm configured to partition the color image into a plurality of clusters of pixels, wherein each pixel in a cluster has a similar color profile; and
determine the energy parameter based on the cluster that includes a pixel associated with the initial location.
